(19) United States
(12) Patent Application Publication
(10) Pub. No.: US 2025/0177376 A1
(43) Pub. Date: Jun. 5, 2025

(54) ALPHA 2 ADRENERGIC RECEPTOR BLOCKADE FOR THE TREATMENT OF C. DIFFICILE COLITIS

(71) Applicant: University of Virginia Patent Foundation, Charlottesville, VA (US)

(72) Inventors: William A. Petri, Jr., Charlottesville, VA (US); David Tyus, Charlottesville, VA (US)

(73) Assignee: University of Virginia Patent Foundation, Charlottesville, VA (US)

(21) Appl. No.: 18/839,296

(22) PCT Filed: Feb. 21, 2023
(86) PCT No.: PCT/US2023/062958

§ 371 Date: Aug. 16, 2024

Related U.S. Application Data
(60) Provisional application No. 63/311,667, filed on Feb. 18, 2022.

(51) Int. Cl. A61K 31/4745; A61K 31/4164; A61K 31/4178; A61K 31/426; A61K 31/437; A61K 31/7048; A61K 38/14; A61K 38/20; A61K 39/395; A61P 31/04

(52) U.S. Cl. CPC A61K 31/4745; A61K 31/4164; A61K 31/4178; A61K 31/426; A61K 31/437; A61K 31/7048; A61K 38/14; A61K 38/20; A61K 38/2026; A61K 38/2086; A61K 39/3955; A61P 31/04

(57) ABSTRACT

Provided are methods and compositions for treating and/or preventing C. difficile infections, particularly recurring C. difficile infections. The compositions for use in treating and/or preventing C. difficile infections include in some embodiments at least one agent that inhibits an alpha 2 adrenergic receptor, and the presently disclosed methods include administering at least one such composition to a subject in need thereof, optionally in combination with other therapeutically active agents including but not limited to an enhancer of an IL-13 biological activity, optionally an IL-13 peptide or a fragment or homolog thereof; an interleukin-13 receptor subunit alpha-2 (IL-13Ra2) inhibitor; an enhancer of an Interleukin-33 (IL-33) biological activity, optionally an IL-33 polypeptide or a biologically active fragment or homolog thereof; or any combination thereof.

### CROSS REFERENCE TO RELATED APPLICATION

The presently disclosed subject matter claims the benefit of U.S. Provisional Patent Application Ser. No. 63/311,667, filed Feb. 18, 2022, the disclosure of which incorporated herein by reference in its entirety.

### GOVERNMENT SUPPORT

This invention was made with government support under Grant Numbers AI152477 and AI124214 awarded by the National Institutes of Health. The government has certain rights in the invention.

### REFERENCE TO SEQUENCE LISTING XML

The Sequence Listing XML associated with the instant disclosure has been electronically submitted to the United States Patent and Trademark Office via the Patent Center as a 43,557 byte UTF-8-encoded XML file created on Feb. 21, 2023 and entitled “3062_178_PCT.xml”. The Sequence Listing submitted via Patent Center is hereby incorporated by reference in its entirety.

### TECHNICAL FIELD

The presently disclosed subject matter relates generally to methods for treating and/or preventing Clostridioides difficile infections and/or consequences thereof, including recurring Clostridioides difficile infections.

### BACKGROUND

Clostridioides difficile (formerly known as Clostridium difficile) is a gram-positive, spore-forming obligate anaerobe. It is the leading cause of hospital acquired gastrointestinal infection in the U.S. The observed rate of C. difficile infection (CDI) has risen from about 5 per 1,000 hospital discharges in 2001 to about 13 per 1,000 discharges in 2011. Risk factors for CDI include age and antibiotic usage. Current CDI therapy relies on antibiotics which greatly increase the risk of recurrent infection.

The sympathetic nervous system (SNS) has emerged as a potent immunomodulator and thus plays a role in CDI pathology. It has been observed that alpha 2 adrenergic receptor (Gene Symbol: ADRA2A), which binds catecholamines norepinephrine and epinephrine, is downregulated in severe CDI as compared to mild CDI. Pharmacological ablation of peripheral SNS neurons that noradrenergic neurons but not dopaminergic neurons are necessary for disease phenotypes (0% mortality vs 100% in control, p<0.0001). Additionally, the immune response (both cellular infiltration and cytokine levels) was greatly diminished in mice lacking peripheral neurons. Blockade of the alpha 2 adrenergic receptor, but not beta adrenergic receptors, greatly improves outcomes in CDI mice (20% mortality vs 100% in control; p<0.0001). Together these data demonstrate that adrenergic signaling through the alpha 2 adrenergic receptor is necessary for CDI disease pathology in mice.

Accordingly, there is an ongoing need for additional methods of treating or preventing C. difficile infections.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides a method for treating and/or preventing a C. difficile infection, and/or a symptom or pathology associated therewith, in a subject in need thereof. In some embodiments, the method comprises, consists essentially of, or consists of administering to the subject a composition that comprises a therapeutically effective amount of at least one agent that inhibits a biological activity of alpha 2 adrenergic receptor in the subject.

In some embodiments, the presently disclosed subject matter relates to methods for treating and/or preventing a Clostridioides difficile (C. difficile) infection (CDI), and/or a symptom or pathology associated therewith, in a subject in need thereof. In some embodiments, the methods comprise, consist essentially of, or consist of administering to the subject a composition comprising, consisting essentially of, or consisting of at least one agent that inhibits an alpha 2 adrenergic receptor (ADRA2A) biological activity in the subject, wherein the administering is in an amount and via a route that treats and/or prevents the C. difficile infection and/or the symptom or pathology associated therewith in the subject. In some embodiments, the at least one agent is selected from the group consisting of RX 821002, yohimbine, mianserin, mirtazapine, esmirtazapine, atipamezole, efaroxan, idazoxan, rauwolscine, spegatrine, dispegatrine, and phentolamine. In some embodiments, the at least one agent is selected from the group consisting of RX 821002 and yohimbine.

In some embodiments, the presently disclosed methods further comprise administering to the subject at least one additional anti-C. difficile agent. In some embodiments, the at least one additional anti-C. difficile agent is selected from the group consisting of an enhancer of an IL-13 biological activity, optionally wherein the enhancer of the IL-13 biological activity comprises, consists essentially of, or consists of an IL-13 peptide or a fragment or homolog thereof; an interleukin-13 receptor subunit alpha-2 (IL-13Ra2) inhibitor; an enhancer of an Interleukin-33 (IL-33) biological activity, optionally an IL-33 polypeptide or a biologically active fragment or homolog thereof; and combinations thereof. In some embodiments, the IL-13Ra2 inhibitor is an antibody that binds to IL-13Ra2, optionally wherein the antibody that binds to IL-13Ra2 is a monoclonal antibody.

In some embodiments, the subject is a mammal, optionally a human. In some embodiments, the human has one or more increased risk factors for CDI, optionally wherein the one or more increased risk factors for CDI are selected from the group consisting of hospitalization, antibiotic use, and increased age.

In some embodiments, the presently disclosed methods, further comprise administering to the subject at least one additional therapeutic agent, optionally wherein the at least one additional therapeutic agent is selected from the group consisting of an anesthetic, an analgesic, an antimicrobial agent, a steroid, a growth factor, a cytokine other than IL-13, and an anti-inflammatory agent. In some embodiments, the at least one additional therapeutic agent comprises a cytokine other than IL-13, optionally an interleukin other than IL-13, further optionally wherein said at least one additional therapeutic agent comprises interleukin-4 (IL-4), interleukin-33 (IL-33), or interleukin-25 (IL-25), or any combination thereof. In some embodiments, the at least one additional therapeutic agent comprises an antimicrobial agent, optionally wherein said antimicrobial agent is selected from the group consisting of an antibacterial agent, an antifungal agent, and an antiviral agent. In some embodiments, the antimicrobial agent comprises at least one antibiotic selected from the group consisting of vancomycin, fidaxomicin, metronidazole, nitazoxanide, and rifaximin.

In some embodiments, the at least one agent that inhibits an ADRA2A biological activity in the subject, the optional at least one additional anti-C. difficile agent, or both are administered at least twice to the subject.

In some embodiments, the symptom or pathology associated with the CDI is selected from the group consisting of weight loss, diarrhea, and colonic inflammation, optionally wherein the colonic inflammation is selected from the group consisting of neutrophil inflammation, monocyte inflammation, and eosinophile inflammation.

The presently disclosed subject matter also relates in some embodiments to compositions for use in treating and/or preventing Clostridioides difficile (C. difficile) infection and/or a symptom and/or pathology associated therewith. In some embodiments, the composition comprises, consists essentially of, or consists of a therapeutically effective amount of at least one agent that inhibits an alpha 2 adrenergic receptor (ADRA2A) biological activity in the subject. In some embodiments, the at least one agent that inhibits the ADRA2A biological activity is selected from the group consisting of RX 821002, yohimbine, mianserin, mirtazapine, esmirtazapine, atipamezole, efaroxan, idazoxan, rauwolscine, spegatrine, dispegatrine, and phentolamine, optionally wherein at least one agent that inhibits the ADRA2A biological activity is selected from the group consisting of RX 821002 and yohimbine. In some embodiments, the composition further comprises a therapeutically effective amount of at least one agent comprising an enhancer of an IL-13 biological activity, optionally wherein the enhancer of the IL-13 biological activity comprises, consists essentially of, or consists of an IL-13 peptide or a fragment or homolog thereof; an interleukin-13 receptor subunit alpha-2 (IL-13Ra2) inhibitor; an enhancer of an Interleukin-33 (IL-33) biological activity, optionally an IL-33 polypeptide or a biologically active fragment or homolog thereof; and combinations thereof. In some embodiments, the IL-13Ra2 inhibitor is an antibody that binds to IL-13Ra2, optionally wherein the antibody that binds to IL-13Ra2 is a monoclonal antibody.

In some embodiments, the compositions methods and compositions of the presently disclosed subject matter reduce mortality, prevents or inhibits recurrent infection, reduces weight loss, reduces diarrhea, and/or reduces colonic inflammation, optionally wherein the colonic inflammation is selected from the group consisting of neutrophil inflammation, monocyte inflammation, and eosinophil inflammation, when administered to a subject in need thereof as compared to what would have occurred had the subject not been administered the at least one agent that inhibits the ADRA2A biological activity or the composition.

Accordingly, it is an object of the presently disclosed subject matter to provide compositions and methods for treating and/or preventing C. difficile infections.

This and other objects are achieved in whole or in part by the presently disclosed subject matter. Further, objects of the presently disclosed subject matter having been stated above, other objects and advantages of the presently disclosed subject matter will become apparent to those skilled in the art after a study of the following Description, Figures, and EXAMPLES. Additionally, various aspects and embodiments of the presently disclosed subject matter are described in further detail below.

### BRIEF DESCRIPTION OF THE FIGURES

FIG. 1 is a graph showing that expression of alpha 2 adrenergic receptors (ADRA2A) is downregulated in severe CDI.

FIG. 2 is an exemplary schematic image of a semi-permanent method for ablating peripheral catecholaminergic neurons (6-OHDA).

FIG. 3 is an exemplary timeline for a making and using a CDI mouse model.

FIGS. 4A to 4B are graphs showing that sympathetic nerve ablation protects from CDI in the mouse model of FIG. 3. FIG. 4A shows probability of survival after sympathetic nerve ablation by 6OHDA (thicker black line) in the mouse model as compared to a vehicle control (thinner black line). FIG. 4B shows weight loss after sympathetic nerve ablation by 6OHDA (squares) in the mouse model as compared to a vehicle control (circles). Error bars represent ±standard error. ***: p<0.001.

FIG. 5 is a graph of probability of survival after sympathetic nerve ablation by 6OHDA (thicker black line) in the mouse model as compared to a PBS control (thinner black line). The data demonstrate that protection is likely not due to specific protection against binary toxin.

FIG. 6 is an exemplary schematic of desipramine sparing noradrenergic neurons from 6-OHDA mediated ablation.

FIGS. 7A-7C show the design and results of experiments demonstrating noradrenergic neurons mediating CDI infection pathogenesis. FIG. 7A is an exemplary timeline of the experiments with a representative mouse CDI model subjected to 80 mg/kg (i.p.) saline+phosphate buffered solution (PBS), or saline+6-OHDA or Desipramine (i.p.)+6-OHDA. FIG. 7B is a graph of the results of weight loss in 6-OHDA+Desipramine (triangles) as compared to saline+6-OHDA alone (squares) and a saline+PBS control (circles). Error bars represent ±standard error. *: p<0.05 and ***: p<0.001. FIG. 7C is a graph showing probability of survival in 6-OHDA+Desipramine (6OHDA_Des) as compared to 6-OHDA alone and a PBS control. For 6-OHDA+Desipramine vs. 6-OHDA alone, the result was statistically significant (p=0.001).

FIGS. 8A-8E are graphs showing that Luminex 32-plex demonstrates hypo-responsive immune systems in 6-OHDA mice. FIGS. 8A-8D are bar graphs showing various cytokine concentrations in 6-OHDA-treated mice (second bar of each pair) versus PBS-treated (i.e., negative control) mice (first bar of each pair). Error bars represent ±standard error. *: p<0.05, **: p<0.01; and ***: p<0.001. FIG. 8E is a heat map of expression levels of cytokines in 6-OHDA-treated mice as compared to PBS-treated (i.e., negative control) mice.

FIGS. 9A-9C are plots showing that noradrenergic neurons are required for CDI-induced recruited immunity. FIG. 9A is a plot showing eosinophil levels in negative control mice (PBS/Saline) vs. 6-OHDA-treated mice (6OHDA) vs. mice treated with 6-OHDA+Desipramine (6OHDA+Des), which indicated that eosinophiles were protective in CDI. FIG. 9B is a plot showing neutrophil levels in negative control mice (PBS/Saline) vs. 6-OHDA-treated mice (6OHDA) vs. mice treated with 6-OHDA+Desipramine (6OHDA+Des), which indicated that neutrophils were complicated in CDI. FIG. 9C is a plot showing monocyte levels in negative control mice (PBS/Saline) vs. 6-OHDA-treated mice (6OHDA) vs. mice treated with 6-OHDA+Desipramine (6OHDA+Des), which indicated that monocytes were protective in CDI.

FIGS. 10A and 10B provide a mouse model and graph of the Beta 1 and 2 blocker propranolol exacerbating CDI disease progression. FIG. 10A shows a representative timeline for creating and testing a mouse CDI model subjected to 10 mg/kg propranolol (i.p.) or PBS (i.p.). FIG. 10B is a graph of percent survival of propranolol-treated mice as compared to negative control (PBS-treated) mice. There was a statistically significant difference in percent survival between the two groups (p=0.002).

FIGS. 11A and 11B provide a mouse model and graph showing that Beta 3 blockade exacerbated CDI disease progression. FIG. 11A shows a representative timeline for creating and testing a mouse CDI model subjected to 10 mg/kg SR 59230A or PBS administered i.p. FIG. 11B is a graph of percent survival of SR 59230A-treated mice as compared to negative control (PBS-treated) mice. There was a statistically significant difference in percent survival between the two groups (p<0.0001).

FIGS. 12A and 12B provide a mouse model and graphs showing that Alpha 2 adrenergic receptor blockade ameliorated CDI induced mortality. FIG. 12A shows a representative timeline for creating and testing a mouse CDI model subjected to 10 mg/kg RX 821002 (i.p.) or PBS (i.p.). FIG. 12B is a graph of survival of RX 821002-treated mice as compared to a negative control (PBS-treated mice). There was a statistically significant difference in percent survival between the two groups (p<0.0001).

FIG. 13 is a graph of survival of mice treated with yohimbine, another alpha 2 adrenergic receptor blocker, as compared to a negative control (PBS treatment). Yohimbine provided a statistically significant amelioration of CDI induced mortality (p=0.0002).

FIG. 14 is a graph showing colonization resulted in no difference in CDI burden. A negative control treatment group (PBS) and an RX 821002 treatment group (RX 821002) showed no difference in colony-forming units (CFU)/mg/mL (p=0.99). Error bars represent ±standard error.

FIG. 15 is a graph showing RX 821002 (RXa) treatment had no effect on CDI colonization as compared to a negative control (PBSa). Error bars represent +standard error.

FIG. 16 is a graph showing that RX 821002 (RX) treatment reduced toxin A/B in CDI cecal content.

FIGS. 17A and 17B are survival graphs and clinical score graphs, respectively, showing that blocking norepinephrine synthesis ameliorated CDI. In FIG. 17A, mice administered the dopamine β-hydroxylase (DBH) inhibitor nepicastat hydrochloride (thicker black line), which blocks norepinephrine synthesis, showed significantly improved survival (p=0.006) as compared to mice administered the ORA-PLUS® vehicle alone (Oraplus; thinner black line). In FIG. 17B, clinical scores calculated as described herein below were also statistically significantly lower (i.e., better) in mice administered nepicastat hydrochloride (thick black line with squares) as compared to mice administered the ORA-PLUS® vehicle alone (Oraplus; thinner black line with circles). Error bars are+standard error. *: p<0.05; **: p<0.01; ***: p<0.0001.

### DETAILED DESCRIPTION

### I. General Considerations

Clostridioides difficile (C. difficile) infection (CDI) is the most common hospital-acquired infection and can manifest in a range of symptoms from diarrheal illness to death. Current CDI therapy relies on antibiotics, which greatly increase the risk of recurrent infection. Thus, the presently disclosed subject matter relates in some embodiments to alternative therapeutic approaches that spare protective gut microbiota, particularly gut microbes that target immune effectors since, as disclosed herein, the immune response is an extremely strong predictor (in humans) and modulator (in mice) of CDI disease severity.

The sympathetic nervous system (SNS) has emerged as a potent immunomodulator. Thus, the role of the SNS in CDI pathology was examined. In mouse ceca, it was initially observed that the alpha 2 adrenergic receptor (by convention, ADRA2A in humans or generically, Adra2a when in reference to non-human species), which binds the catecholamines norepinephrine and epinephrine, was downregulated in severe CDI as compared to mild CDI. It was further found through pharmacological ablation of peripheral SNS neurons that noradrenergic neurons but not dopaminergic neurons were necessary for disease phenotypes (0% mortality vs 100% in control; p<0.0001). Additionally, the immune response (both cellular infiltration and cytokine levels) was greatly diminished in mice lacking peripheral neurons. It was also found that blockade of the alpha 2 adrenergic receptor, but not beta adrenergic receptors, greatly improves outcomes in CDI mice (20% mortality vs 100% in control; p <0.0001). Together these data demonstrate that adrenergic signaling through the alpha 2 adrenergic receptor is necessary for CDI disease pathology in mice. This is the first study in CDI research to demonstrate a role of neurons in CDI in vivo. Additional examples assess whether the alpha 2 adrenergic receptor drives immune responses (specifically neutrophil infiltration) in CDI to onset disease. In these examples, adrenergic influence of CDI pathology is validated through genetic models and it is determined which cells expressing alpha 2 adrenergic receptor play a role in the phenotypes observed. These examples not only highlight neuroimmune pathways as potential therapeutic targets in CDI but also allow for the identification of neuromodulators such as antidepressants that may either be predisposing for CDI or serve as therapies to be repurposed for CDI patients.

An aspect of the presently disclosed subject matter pertains to the discovery in a mouse model of C. difficile colitis that noradrenergic neurons mediate C. difficile infection pathogenesis. Sympathetic nerve ablation with 6-hydroxydopamine provided complete protection from death in C57BI/6J mice challenged with the epidemic strain of C. difficile R20297. Protection was associated with a block in pro-inflammatory cytokine response to infection and a reduction in recruitment to the colon of neutrophils, monocytes, and eosinophils. The adrenergic receptor responsible for mediating the noradrenergic exacerbation of infection was identified as the alpha 2 receptor by protection by alpha 2 blockade with the drug RX 821002, in an exemplary embodiment.

The presently disclosed subject matter will now be described more fully. The presently disclosed subject matter can, however, be embodied in different forms and should not be construed as limited to the embodiments set forth herein below and in the accompanying EXAMPLES. Rather, these embodiments are provided so that this disclosure will be thorough and complete, and will fully convey the scope of the embodiments to those skilled in the art.

### II. Definitions

All references cited in the specification are incorporated herein by reference to the extent that they supplement, explain, provide a background for and/or teach methodology, techniques and/or compositions employed herein. All cited patents and publications referred to in this application are herein expressly incorporated by reference.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “a therapeutic agent” refers to one or more therapeutic agents, e.g., one or more of the same of different therapeutic agents. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of time, concentration percent inhibition, percent viability, amounts of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments +5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” unless stated otherwise.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

As used herein, the phrase “alpha-2-adrenergic receptor”, also known as “α2-adrenergic receptor” and “adrenoceptor alpha 2A” and by the abbreviations “ADRA2A”, “ADRAR”, “ZNF32”, “ADRA2R”, and “ALPHA2AAR”, refers to genetic loci and nucleic acids and polypeptides encoded thereby that are particular members of the G protein-coupled receptor superfamily that inhibit adenylate cyclase. Exemplary ADRA2A orthologs include the nucleotide and amino acid sequences as set forth in the GENBANK® biosequence database and summarized as follows.

| Accession No. | Description |
|---|---|
| NC_000010.11 | Human genomic locus; chromosome 11, nucleotides |
| | 111,077,029-111,080,907 |
| NM_000681.4 | Human adrenoceptor alpha 2A (ADRA2A), mRNA; |
| | SEQ ID NO; 1 |
| NP_000672.3 | Human alpha-2A adrenergic receptor polypeptide |
| | encoded by NM_000681.4; SEQ ID NO; 2 |
| NC_000085.7 | Mouse genomic locus; chromosome 19, nucleotides |
| | 54,033,690-54,037,413 |
| NM_007417.5 | Mus musculus adrenergic receptor, alpha 2a |
| | (ADRA2A), mRNA; SEQ ID NO; 3 |
| NP_031443.4 | Mus musculus adrenergic receptor, alpha 2a |
| | (ADRA2A) polypeptide encoded by NM_007417.5; |
| | SEQ ID NO; 4 |
| NM_012739.3 | Rattus norvegicus adrenoceptor alpha 2A (ADRA2A), |
| | mRNA; SEQ ID NO; 5 |
| NP_036871.3 | Rattus norvegicus adrenoceptor alpha 2A (ADRA2A) |
| | polypeptide encoded by NM_012739.3; SEQ ID NO; 6 |
| XM_003312765.3 | Pan troglodytes adrenoceptor alpha 2A (ADRA2A), |
| | mRNA; SEQ ID NO; 7 |
| XP_003312813.2 | Pan troglodytes adrenoceptor alpha 2A (ADRA2A) |
| | encoded by XM_003312765.3; SEQ ID NO; 8 |

As used herein, the phrases “alpha-2-adrenergic receptor inhibitor”, “ADRA2A inhibitor”, and “alpha 2 antagonist” are used interchangeably and refer to a molecule that causes inhibition of an ADRA2A biological activity. ADRA2A inhibitors include but are not limited to small molecule inhibitors, nucleic acids that inhibit a biological activity of an ADRA2A gene product, and antibodies that inhibit a biological activity of an ADRA2A gene product or another gene product that acts downstream of an ADRA2A gene product. Exemplary ADRA2A inhibitors include RX 821002, yohimbine, mianserin, mirtazapine, esmirtazapine, atipamezole, efaroxan, idazoxan, rauwolscine, spegatrine, dispegatrine, and phentolamine.

A pathology or symptom “associated” with C. difficile infection (CDI) refers to mortality, colonic inflammation, diarrhea, weight loss, changes in expression and levels of genes, proteins, and cells as described herein or those that are known in the art that occur upon CDI and/or are a consequence of CDI.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

As used herein, a “derivative” of a compound refers to a chemical compound that can be produced from another compound of similar structure in one or more steps, as in replacement of H by an alkyl, acyl, or amino group.

As used herein, the term “desipramine” refers to the norepinephrine reuptake inhibitor 3-(10,11-dihydro-5H-dibenzo[b,f]azepin-5-yl)-N-methylpropan-1-amine. It corresponds to CAS Number 50-47-5.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, the terms “condition”, “disease condition”, “disease”, “disease state”, and “disorder” refer to physiological states in which diseased cells or cells of interest can be targeted with the compositions of the presently disclosed subject matter.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, can be different from when that compound is administered alone. Thus, an effective amount of a combination of compounds refers collectively to the combination as a whole, although the actual amounts of each compound can vary. The term “more effective” means that the selected effect occurs to a greater extent by one treatment relative to the second treatment to which it is being compared.

As used herein, “homology” is used synonymously with “identity.” As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term nepicastat refers to the selective dopamine β-hydroxylase (DBH) inhibitor 5-(Aminomethyl)-1-[(2S)-5,7-difluoro-1,2,3,4-tetrahydro-2-naphthalenyl]-1,3-dihydro-2H-imidazole-2-thione. Its hydrochloride form corresponds to CAS Number 170151-24-3.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

In some embodiments, a “preventive” or “prophylactic” treatment is a treatment administered to a subject who does not exhibit signs, or exhibits only early signs, of a condition, disease, or disorder. Thus, a prophylactic or preventative treatment can be administered for the purpose of decreasing the risk of developing pathology associated with developing the condition, disease, or disorder.

The term “nucleic acid” refers to deoxyribonucleotides or ribonucleotides and polymers thereof in either single or double stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed base and/or deoxyinosine residues. The terms “nucleic acid” or “nucleic acid sequence” can also be used interchangeably with gene, open reading frame (ORF), cDNA, and mRNA encoded by a gene.

The term “RNA” refers to a molecule comprising at least one ribonucleotide residue. By “ribonucleotide” is meant a nucleotide with a hydroxyl group at the 2′ position of a β-D-ribofuranose moiety. The terms encompass double stranded RNA, single stranded RNA, RNAs with both double stranded and single stranded regions, isolated RNA such as partially purified RNA, essentially pure RNA, synthetic RNA, recombinantly produced RNA, as well as altered RNA, or analog RNA, that differs from naturally occurring RNA by the addition, deletion, substitution, and/or alteration of one or more nucleotides. Such alterations can include addition of non-nucleotide material, such as to the end(s) of an siRNA or internally, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

The terms “short hairpin RNA” and “shRNA” are used interchangeably and refer to any nucleic acid molecule capable of generating siRNA. In one embodiment, the shRNA comprises a polynucleotide having one or more loop structures and a stem comprising self complementary sense and antisense regions, wherein the antisense region comprises a sequence complementary to a region of a target nucleic acid molecule, and wherein the polynucleotide can be processed either in vivo or in vitro to generate an active siRNA capable of mediating RNAi. In another embodiment, retroviral vectors encode shRNA, which are processed intracellularly, to generate siRNA that silence the expression of a target gene, such as a gene encoding an ADRA2A gene product.

The terms “small interfering RNA”, “short interfering RNA” and “siRNA” are used interchangeably and refer to any nucleic acid molecule capable of mediating RNA interference (RNAi) or gene silencing. See e.g., Bass (2001) RNA interference. The short answer. Nature 411:428-429; Elbashir et al. (2001) Duplexes of 21-nucleotide RNAs mediate RNA interference in cultured mammalian cells. Nature 411:494-498; Fire et al. (1998) Potent and specific genetic interference by double-stranded RNA in Caenorhabditis elegans. Nature 391:806-811; and PCT International Publication Nos. WO 2000/44895, WO 2001/36646, WO 1999/32619, WO 2000/01846, WO 2001/29058, WO 1999/07409, and WO 2000/44914. In one embodiment, the siRNA comprises a double stranded polynucleotide molecule comprising complementary sense and antisense regions, wherein the antisense region comprises a sequence complementary to a region of a target nucleic acid molecule (for example, an mRNA encoding an ADRA2A gene product). In some embodiments, the siRNA comprises a single stranded polynucleotide having self-complementary sense and antisense regions, wherein the antisense region comprises a sequence complementary to a region of a target nucleic acid molecule. As used herein, siRNA molecules need not be limited to those molecules containing only RNA, but further encompass chemically modified nucleotides and non-nucleotides.

The terms “microRNA” and “miRNA” refer are used interchangeably and refer to synthetic or single-stranded RNA molecules of 21-23 nucleotides in length, which regulate gene expression. The terms “miRNA” and “non-coding RNA” can be used interchangeably. Primary transcript (pri-miRNA) is processed to give rise to short-stem-loop pre-miRNA, which are further processed to produce miRNA, which are single-stranded RNA molecules of 21-23 nucleotides. The miRNA are partially complementary to one or several mRNA transcripts, and they downregulate expression of genes encoded by the transcripts with which they interact. Thus, by way of example and not limitation, synthetic miRNA that interact with an ADRA2A gene productcan be generated and used to effect the downregulation of ADRA2A expression thereby inhibiting ADRA2A protein expression and consequently ADRA2A biological activity.

The term “Ribozyme”, also known as “RNA enzyme” or “catalytic RNA” refers to ribonucleotides or RNA molecules that can act as enzymes that catalyze covalent changes in the structure of RNA molecules and that can cleave the target RNA molecule. Synthetic hammerhead ribozymes can be generated that recognize and cleave ADRA2A RNA, or any other target disclosed herein, thereby inhibiting ADRA2A protein expression.

The terms “identical” or percent “identity” in the context of two or more nucleotide or polypeptide sequences refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term “substantially identical”, in the context of two nucleotide sequences, refers to two or more sequences or subsequences that have in some embodiments at least 60%, in some embodiments about 70%, in some embodiments about 80%, in some embodiments about 90%, in some embodiments about 95%, in some embodiments, about 97%, and in some embodiments about 99% nucleotide identity, when compared and aligned for maximum correspondence, as measured using one of the following sequence comparison algorithms (described herein below) or by visual inspection. In some embodiments, the substantial identity exists in nucleotide sequences of at least 50 residues, in some embodiments in nucleotide sequence of at least about 100 residues, in some embodiments in nucleotide sequences of at least about 150 residues, and in some embodiments in nucleotide sequences comprising complete coding sequences.

In some embodiments, polymorphic sequences can be substantially identical sequences. The terms “polymorphic”, “polymorphism”, and “polymorphic variants” refer to the occurrence of two or more genetically determined alternative sequences or alleles in a population. An allelic difference can be as small as one base pair. As used herein with respect to a nucleotide or polypeptide sequence, the term “substantially identical” also refers to a particular sequence that varies from another sequence by one or more deletions, substitutions, or additions, the net effect of which is to retain biological activity of a gene, gene product, or sequence of interest.

For sequence comparison, typically one sequence acts as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, e.g., by the local homology algorithm of Smith & Waterman (1981) 2 Adv Appl Math 482-489; by the homology alignment algorithm disclosed in Needleman & Wunsch (1970) A general method applicable to the search for similarities in the amino acid sequence of two proteins. 48 J Mol Biol 443-453; by the search for similarity method disclosed in Pearson & Lipman (1988) Improved tools for biological sequence comparison. Proc Natl Acad Sci USA 85:2444-2448; by computerized implementations of these algorithms (GAP, BESTFIT, FASTA, and TFASTA in the GCG® WISCONSIN PACKAGE®, available from Accelrys, Inc., San Diego, California, United States of America), or by visual inspection. See generally, Altschul et al. (1990) Basic local alignment search tool. J Mol Biol 215:403-410; Ausubel et al. (2002) Short Protocols in Molecular Biology, Fifth ed., Wiley, New York, New York, United States of America; and Ausubel et al. (2003) Current Protocols in Molecular Biology, John Wylie & Sons, Inc, New York, New York, United States of America.

In some embodiments, an algorithm for determining percent sequence identity and sequence similarity is the BLAST algorithm, which is described by Altschul et al. (1990) Basic local alignment search tool. J Mol Biol 215:403-410. Software for performing BLAST analyses is publicly available through the website of the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff (1992) Amino acid substitution matrices from protein blocks. Proc Natl Acad Sci USA 89 (22):10915-10919.

In addition to calculating percent sequence identity, the BLAST algorithm also performs a statistical analysis of the similarity between two sequences. See e.g., Karlin & Altschul (1993) Applications and statistics for multiple high-scoring segments in molecular sequences. Proc Natl Acad Sci USA 90:5873-5877. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in some embodiments less than about 0.1, in some embodiments less than about 0.01, and in some embodiments less than about 0.001.

Another indication that two nucleotide sequences are substantially identical is that the two molecules specifically or substantially hybridize to each other under stringent conditions. In the context of nucleic acid hybridization, two nucleic acid sequences being compared can be designated a “probe” and a “target”. A “probe” is a reference nucleic acid molecule, and a “'target” is a test nucleic acid molecule, often found within a heterogeneous population of nucleic acid molecules. A “target sequence” is synonymous with a “test sequence”.

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

“Stringent hybridization conditions” and “stringent hybridization wash conditions” in the context of nucleic acid hybridization experiments such as Southern and Northern blot analysis are both sequence-and environment-dependent. Longer sequences hybridize specifically at higher temperatures. An extensive guide to the hybridization of nucleic acids is found in Tijssen (1993) Laboratory Techniques in Biochemistry and Molecular Biology. van der Vliet (ed.), vol. 24, Hybridization with nucleic acid probes, Elsevier, Amsterdam, The Netherlands. Generally, highly stringent hybridization and wash conditions are selected to be about 5° C. lower than the thermal melting point (Tm) for the specific sequence at a defined ionic strength and pH. Typically, under “stringent conditions” a probe will hybridize specifically to its target subsequence, but to no other sequences.

The Tm is the temperature (under defined ionic strength and pH) at which 50% of the target sequence hybridizes to a perfectly matched probe. Very stringent conditions are selected to be equal to the Tm for a particular probe. An example of highly stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1× standard saline citrate (SSC), 0.1% (w/v) SDS at 65° C. Another example of highly stringent wash conditions is 15 minutes in 0.2× SSC buffer at 65° C. (see Green & Sambrook (2012) Molecular Cloning: A Laboratory Manual (Fourth Edition). Cold Spring Harbor Laboratory Press, Cold Spring Harbor Laboratory, New York, United States of America for a description of SSC buffer and other stringency conditions). Often, a high stringency wash is preceded by a lower stringency wash to remove background probe signal. An example of medium stringency wash conditions for a duplex of more than about 100 nucleotides is 15 minutes in 1× SSC at 45° C. Another example of medium stringency wash for a duplex of more than about 100 nucleotides is 15 minutes in 4-6× SSC at 40° C. For short probes (e.g., about 10 to 50 nucleotides), stringent conditions typically involve salt concentrations of less than about 1M Na+ ion, typically about 0.01 to 1M Na+ ion concentration (or other salts) at pH 7.0-8.3, and the temperature is typically at least about 30° C. Stringent conditions can also be achieved with the addition of destabilizing agents such as formamide. In general, a signal to noise ratio of 2-fold or higher than that observed for an unrelated probe in the particular hybridization assay indicates detection of a specific hybridization.

The following are examples of hybridization and wash conditions that can be used to clone homologous nucleotide sequences that are substantially identical to reference nucleotide sequences of the presently disclosed subject matter: a probe nucleotide sequence hybridizes in one example to a target nucleotide sequence in 7% sodium dodecyl sulfate (SDS), 0.5M NaPO₄, 1 mm EDTA at 50° C. followed by washing in 2× SSC, 0.1% SDS at 50° C.; in another example, a probe and target sequence hybridize in 7% sodium dodecyl sulfate (SDS), 0.5M NaPO₄, 1 mm EDTA at 50° C. followed by washing in 1× SSC, 0.1% SDS at 50° C.; in another example, a probe and target sequence hybridize in 7% sodium dodecyl sulfate (SDS), 0.5M NaPO₄, 1 mm EDTA at 50° C. followed by washing in 0.5× SSC, 0.1% SDS at 50° C.; in another example, a probe and target sequence hybridize in 7% sodium dodecyl sulfate (SDS), 0.5M NaPO₄, 1 mm EDTA at 50° C. followed by washing in 0.1× SSC, 0.1% SDS at 50° C.; in yet another example, a probe and target sequence hybridize in 7% sodium dodecyl sulfate (SDS), 0.5M NaPO₄, 1 mm EDTA at 50° C. followed by washing in 0.1× SSC, 0.1% SDS at 65° C.

The term “similarity” is contrasted with the term “identity”. Similarity is defined as above; “identity”, however, means a nucleic acid or amino acid sequence having the same amino acid at the same relative position in a given family member of a gene family. Homology and similarity are generally viewed as broader terms than the term identity. Biochemically similar amino acids, for example leucine and isoleucine or glutamate/aspartate, can be present at the same position. These are not identical per se, but are biochemically “similar”. As disclosed herein, these are referred to as conservative differences or conservative substitutions. This differs from a conservative mutation at the DNA level, which changes the nucleotide sequence without making a change in the encoded amino acid, e.g. TCC to TCA, both of which encode serine.

Nucleotide sequences are “substantially identical” where they have in some embodiments between about 70% and about 80%, in some embodiments between about 81% and about 90%, or in some embodiments between about 91% and about 99%, sequence identity for nucleic acid residues which are identical to a reference nucleotide sequence (e.g., an ADRA2A gene product of the presently disclosed subject matter).

Peptide sequences which have about in some embodiments 35%, in some embodiments 45%, in some embodiments from 45-55%, in some embodiments 55-65%, in some embodiments 65% or greater, in some embodiments 70%, in some embodiments about 80%; in some embodiments between about 81% and about 90%; in some embodiments between about 91% and about 99% (e.g., 91%, 92%, 93%, 94%, and 95%) amino acids which are identical or functionally equivalent or biologically functionally equivalent to the amino acids of an ADRA2A gene product of the presently disclosed subject matter are sequences which are “substantially similar”.

The term “homology” describes a mathematically based comparison of sequence similarities which is used to identify genes or proteins with similar functions or motifs. Accordingly, the term “homology” is synonymous with the term “similarity” and “percent similarity” as defined above. Thus, the phrases “substantial homology” or “substantial similarity” have similar meanings.

The term “SR 59230A” refers to (2S)-1-(2-ethylphenoxy)-3-[[(1S)-1,2,3,4-tetrahydronaphthalen-1-yl]amino]propan-2-ol, which is a selective beta-3 adrenergic receptor antagonist. It corresponds to CAS Number 174689-39-5.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichthyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, a “subject” or “subject in need thereof” is a patient, animal, mammal, or human, who will benefit from the method of the presently disclosed subject matter.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented. The term “treating” refers any effect, e.g., lessening, reducing, modulating, ameliorating, reversing, or eliminating, that results in the improvement of the condition, disease, disorder, and the like, or ameliorating a symptom thereof.

### II. Compositions and Methods of the Presently Disclosed Subject Matter

C. difficile Infection (CDI) is the leading hospital-acquired gastrointestinal infections and contributes to a significant burden in the healthcare industry. While the bacterial toxins and their contributions to pathogenesis have been studied extensively, the impact of the robust inflammatory immune response to disease is not well understood.

Accordingly, in some embodiments, the presently disclosed subject matter provides compositions and methods for treating and/or preventing a C. difficile infection, or a pathology or symptom associated therewith, in a subject in need thereof by administering to said subject a composition comprising a therapeutically effective amount of at least one agent that of at least one agent that inhibits a biological activity of alpha 2 adrenergic receptor (ADRA2A) in the subject.

### II.A. Compositions

The compositions of the presently disclosed subject matter comprise, consist essentially of, or consist of one or more ADRA2A inhibitors. Any ADRA2A inhibitor can be employed in the compositions and methods of the presently disclosed subject matter. By way of example and not limitation, an ADRA2A inhibitor can be a small molecule inhibitor, a nucleic acid that inhibits a biological activity of an ADRA2A gene product, an antibody that inhibits a biological activity of an ADRA2A gene product or another gene product that acts downstream of an ADRA2A gene product, etc.

Various small molecule ADRA2A inhibitors are known. Exemplary small molecule ADRA2A inhibitors include, but are not limited to the following 2-(3-methoxy-2H-1,4-benzodioxin-3-yl)-4,5-dihydro-1H-imidazole (RX 821002, which is also called RX821002 and 2-methoxyidazoxan; CAS Number 102575-24-6); 5-(2-ethyl-1,3-dihydroinden-2-yl)-1H-imidazole (Atipamezole, also called antisedan; CAS Number 104054-27-5); methyl (1S,15R,18S,19R,20S)-18-hydroxy-1,3,11,12, 14,15,16,17,18,19,20,21-dodecahydroyohimban-19-carboxylate (yohimbine, also called quebrachine: CAS Number 146-48-5); 3-[N-(4,5-dihydro-1H-imidazol-2-ylmethyl)-4-methylanilino]phenol (phentolamine, also called Regitine, Regitin, and fentolamin; CAS Number 50-60-2); 2-(2-ethyl-3H-1-benzofuran-2-yl)-4,5-dihydro-1H-imidazole (Efaroxan; CAS Number 89197-32-0); 2-(2,3-dihydro-1,4-benzodioxin-3-yl)-4,5-dihydro-1H-imidazole (Idazoxan, also called idazoxane: CAS Number 79944-58-4); methyl (1S,15S,18S,19S,20S)-18-hydroxy-1,3,11,12, 14,15,16,17,18,19,20,21-dodecahydroyohimban-19-carboxylate (Rauwolscine, also called alpha-yohimbine, corynanthidine, and isoyohimbine: CAS Number 131-03-3); (1S,13R,14S,15E)-15-ethylidene-13-(hydroxymethyl)-17-methyl-3-aza-17-azoniapentacyclo[12.3.1.02,10.04,9.012,17]octadeca-2(10),4(9),5,7-tetraen-7-ol (spegatrine and its dimer dispegatrine: CAS Number 47326-53-4); 5-methyl-2,5-diazatetracyclo[13.4.0.02,7.08,13]nonadeca-1(19),8,10,12,15,17-hexaene (mianserin, also called mianserine): CAS Number 21535-47-7 (mono-hydrochloride); 5-methyl-2,5,19-zriazatetracyclo[13.4.0.02,7.08,13]nonadeca-1(15), 8,10,12,16,18-hexaene (mirtazapine, also called remeron: CAS Number 85650-52-8); and (7S)-5-methyl-2,5,19-triazatetracyclo[13.4.0.02,7.08,13]nonadeca-1(15),8,10,12,16,18-hexaene (esmirtazapine, also called (S)-Mirtazapine: CAS Number 61337-87-9).

Nucleic acids that inhibit ADRA2A gene biological activities include, but are not limited to inhibitory RNAs. Exemplary inhibitory RNAs include small interfering RNAs (siRNAs). By “small interfering RNAs (siRNAs)” is meant, inter alia, an isolated dsRNA molecule comprised of both a sense and an anti-sense strand. In some embodiments, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. RNA interference is a commonly used method to regulate gene expression. This effect is often achieved by using small interfering RNA or short hairpin RNA (shRNA). Inhibitory RNAs can be designed against ADRA2A gene products (e.g., transcription products including heterogenous nuclear RNAs (hnRNAs) and spliced derivatives thereof, including messenger RNAs (mRNAs)). The nucleotide sequences of exemplary ADRA2A mRNAs are present in the GENBANK® biosequence database, some of which are also provided in Table 1 above (e.g., NM_000681.4 (human; SEQ ID NO: 1), NM_007417.5 (mouse; SEQ ID NO; 3), NM_012739.3 (rat; SEQ ID NO; 5), and XM_003312765.3 (chimpanzee; SEQ ID NO; 7)).

ADRA2A inhibitors also include antibodies and antibody fragments that bind to ADRA2A or downstream members of an ADRA2A biological pathway such as but not limited to anti-ADRA2A humanized antibodies, monoclonal antibodies, and fragments thereof; aptamer, phylomer, proteins that interact with ADRA2A and/or downstream members of an ADRA2A biological pathway to inhibit a biological activity of said ADRA2A or ADRA2A biological pathway. Anti-ADRA2A antibodies are commercially available ThermoFischer from Scientific (Waltham, Massachusetts, United States of America; e.g., INVITROGEN® Catalog Nos. PA1-048, PA5-85077, PA5-120835, PA5-18475, 703636, and MA5-32570; Proteintech Catalog No. 14266-1-AP), Millipore Sigma (Burlington, Massachusetts, United States of America; Catalog No. SAB4500549-100UG), Bio-Techne, formerly R&D Systems (Minneapolis, Minnesota, United States of America; Catalog No. MAB10129), and others.

### II.A.1. Pharmaceutical Compositions

In some embodiments, the compositions of the presently disclosed subject matter are provided as part of a pharmaceutical composition. As used herein, the term “pharmaceutical composition” refers to a composition comprising at least one active ingredient (e.g., an ADRA2A inhibitor of the presently disclosed subject matter), whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

In some embodiments, a pharmaceutical composition of the presently disclosed subject matter comprises, consists essentially of, or consists of at least one active ingredient (e.g., an ADRA2A inhibitor of the presently disclosed subject matter) and a pharmaceutically acceptable diluent and/or excipient. As used herein, the term “pharmaceutically acceptable” refers to physiologically tolerable, for either human or veterinary application. Similarly, “pharmaceutical compositions” include formulations for human and veterinary use. The term “pharmaceutically acceptable carrier” also refers to a chemical composition with which an appropriate compound or derivative can be combined and which, following the combination, can be used to administer the appropriate compound to a subject. In some embodiments, a pharmaceutically acceptable diluent and/or excipient is pharmaceutically acceptable for use in a human.

The pharmaceutical compositions of the presently disclosed subject matter can in some embodiments consist of the active ingredient alone, in a form suitable for administration to a subject, or the pharmaceutical composition can in some embodiments comprise or consist essentially of the active ingredient and one or more pharmaceutically acceptable carriers, one or more additional ingredients, or some combination of these. The active ingredient can be present in the pharmaceutical composition in the form of a physiologically acceptable ester or salt, such as in combination with a physiologically acceptable cation or anion, as is well known in the art.

As used herein, the term “physiologically acceptable” ester or salt refers to an ester or salt form of the active ingredient which is compatible with any other ingredients of the pharmaceutical composition, which is not deleterious to the subject to which the composition is to be administered.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single-or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts.

### II.A.2. Formulations

The compositions of the presently disclosed subject matter thus comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject.

For example, suitable formulations can include aqueous and non-aqueous sterile injection solutions that can contain antioxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, cytokines and other immunomodulating compounds.

Controlled- or sustained-release formulations of a pharmaceutical composition of the presently disclosed subject matter can be made using conventional technology. A formulation of a pharmaceutical composition of the invention suitable for oral administration can be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the presently disclosed subject matter which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin.

Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose, etc. Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively).

Known emulsifying agents include, but are not limited to, lecithin and acacia. Known preservatives include, but are not limited to, methyl, ethyl, or n-propyl parahydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the presently disclosed subject matter can also be prepared, packaged, or sold in the form of oil in water emulsion or a water-in-oil emulsion.

The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the presently disclosed subject matter can also be prepared, packaged, or sold in a formulation suitable for parenteral administration, including but not limited to intraocular injection.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane dial, for example.

Other acceptable diluents and solvents include, but are not limited to, Ringer's solution, isotonic sodium chloride solution, and fixed oils such as synthetic mono or di-glycerides. Other parentally administrable formulations which are useful include those which comprise the active ingredient in microcrystalline form, in a liposomal preparation, or as a component of a biodegradable polymer systems.

Compositions for sustained release or implantation can in some embodiments comprise pharmaceutically acceptable polymeric or hydrophobic materials such as an emulsion, an ion exchange resin, a sparingly soluble polymer, or a sparingly soluble salt. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the presently disclosed subject matter can be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, can in some embodiments have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro (1985) Remington's Pharmaceutical Sciences, Mack Publishing Co., Easton, Pennsylvania, United States of America, which is incorporated herein by reference in its entirety.

### II.A.3. Administration

With regard to administering a composition of the presently disclosed subject matter, methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, subcutaneous administration, intravitreous administration, including via intravitreous sustained drug delivery device, intracameral (into anterior chamber) administration, suprachoroidal injection, subretinal administration, subconjunctival injection, sub-tenon administration, peribulbar administration, transscleral drug delivery, intraocular injection, intravenous injection, intraparenchymal/intracranial injection, intra-articular injection, retrograde ureteral infusion, intrauterine injection, intratesticular tubule injection, intrathecal injection, intraventricular (e.g., inside cerebral ventricles) administration, administration via topical eye drops, and the like. Administration can be continuous or intermittent. In some embodiments, a preparation can be administered therapeutically; that is, administered to treat an existing disease or condition. In some embodiments, a preparation can be administered prophylactically; that is, administered for prevention of a disease, disorder, or condition.

### II.A.4. Dose

An effective dose of a composition of the presently disclosed subject matter is administered to a subject in need thereof. A “treatment effective amount” or a “therapeutic amount” is an amount of a therapeutic composition sufficient to produce a measurable response (e.g., a biologically or clinically relevant response in a subject being treated). Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved. The potency of a composition can vary, and therefore a “treatment effective amount” can vary. However, using the assay methods described herein, one skilled in the art can readily assess the potency and efficacy of a candidate compound of the presently disclosed subject matter and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular disease treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

For example, the number of doses of an alpha 2 antagonist to be administered can also vary depending on the age, sex, weight, body surface area (BSA), and health of the subject, as well as the specific sign(s) of the infection being treated. A pharmaceutical composition of the presently disclosed subject matter can be administered, for example, 1, 2, 3, 4, 5, 6, 7, 8, 9, or 10 times. In some embodiments, it can be administered up to 20 times. In some embodiments, it is administered once daily. In some embodiments it is administered more than once in a day. In some embodiments, it is administered once per week. In some embodiments, it is administered more than once per week. In some embodiments, it is administered once per month. In some embodiments, it is administered more than once per month.

When used in vivo for therapy, the ADRA2A inhibitors (e.g., small molecules, inhibitory nucleic acids, and/or antibodies) of the presently disclosed subject matter are administered to the subject in a therapeutically effective amount (i.e., an amount that has a desired therapeutic effect). Typically, the ADRA2A inhibitors can be administered parenterally. The dose and dosage regimen can depend upon the degree of the infection, the characteristics of the particular ADRA2A inhibitor(s) used, e.g., its therapeutic index, the patient, and the patient's history. In some embodiments, the ADRA2A inhibitors are administered repeatedly and/or continuously over a period of 1-2 weeks, or depending on response, longer time frames can be employed as needed.

In some embodiments, an effective dose of the ADRA2A inhibitor is one that is sufficient to treat infection and/or control diarrhea and/or weight loss in a subject infected with C. difficile. Thus, in some embodiments, the administering inhibits diarrhea e.g., eliminates or reduces diarrhea compared to what would be expected in the absence of the administering. In some embodiments, the administering results inhibits weight loss, e.g., eliminates or reduces weight loss compared to what would be expected in the absence of the administering. In some embodiments, the administering inhibits colonic inflammation, e.g., eliminates or reduces colonic inflammation compared to what would be expected in the absence of the administering or the level of inflammation prior to the administering. In some embodiments, intestinal gut flora is preserved (e.g., such that recurrent disease is prevented).

In some embodiments, an effective dose is a dose that reduces mortality.

In some embodiments, the compositions and methods of the presently disclosed subject matter are useful for preventing relapse in an already treated subject and in preventing reinfection, as well as reducing the frequency of relapse or reinfection.

In some embodiments, the subject in need of treatment is a mammal. In some embodiments, the subject is a human. In some embodiments, the subject is a subject who has a C. difficile infection or a subject who has one or more increased risk factors for a C. difficile infection. Increased risk factors for getting a C. difficile infection include, but are not limited to, hospitalization (current or recent hospitalization, such as hospitalization in the last one or two weeks), antibiotic use (current or recent antibiotic use), and increased age. For example, humans aged 45 years or older have higher risk for CDI than younger adult humans, and risk continues to increase with age. Thus, for example, the subject with increased risk can be a human that is over 45 years of age, over 55 years of age, or over 65 years of age.

One of ordinary skill in the art will appreciate that, the dose of an alpha 2 antagonist can be varied depending on such things as the age, health, sex, and age of the subject as well as the severity of the CDI or whether it is being used as a preventative. Additionally, depending on various parameters regarding the subject, whether a dose is provided in one administration to a subject or as multiples, the presently disclosed subject matter further encompasses doses of about 1.0 μg/kg body weight to about 500 μg/kg body weight. In some embodiments, the range is about 2.0 to about 150 μg/kg body weight. In some embodiments, the range is about 5.0 to about 100 μg/kg body weight. In yet some embodiments, the dose range is about 10 to about 75 μg/kilo body weight. In some embodiments, the range is about 20 to about 50 g/kg body weight. The doses include fractions and decimals of the doses provided herein. In some embodiments, the therapeutically effective dose used is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 58, 59, 60, 61, 62, 62.5, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 μg/kg body weight, and decimals thereof. The total amount to be administered during a day can be divided into lower doses and administered at multiple times/day. In some embodiments, the method is useful for low dose treatment.

In some embodiments, for short durations of treatment the presently disclosed subject matter provides a dosage range of at least one antibiotic of about 0.1 mg/kg to about 75 mg/kg. In some embodiments, it is from about 0.5 to about 50 mg/kg. In some embodiments, it is from about 2.0 to about 40 mg/kg. In some embodiments, it is from about 3.0 to about 35 mg/kg. In some embodiments, it is from about 4.0 to about 30 mg/kg. In a some embodiments, it is from about 5.0 to about 25 mg/kg. In some embodiments, and in some embodiments it is from about 6.0 to about 20 mg/kg. In some embodiments, it is from about 7.0 to about 15 mg/kg.

In some embodiments, the dose is about 0.1, 0.5, 1.0, 2.0, 3.0, 4.0, 5.0, 6.0, 7.0, 8.0, 9.0, 10, 15, 20, 25, 30, 32, 35, 40, 45, 55, 60, 65, 70, or about 75 mg/kg. One of ordinary skill in the art can determine which dose to use depending on whether the treatment is for a short duration, or for a low dose, or a combination of the two. In some embodiments, a short-term treatment such as 1 or 2 days, can use a slightly higher dose than a treatment that lasts longer.

The total amount to be administered during a day can be divided into lower doses and administered at multiple times/day. In some embodiments, the method is useful for low dose treatment. In some embodiments, the method is useful for short-term treatment. For example, if 20 mg/kg/day is the prescribed amount for the day, that amount can be divided into more than one dose for administration during the day, such as doses of 10 mg/kg administered twice. In some embodiments, treatment can be as short as 1 day. In some embodiments, even doses as low as 0.01, 0.1, 0.5, 1.0, 1.5, 2.0, 2.5, 3.0, 3.5, 4.0, 4.5, and 5.0 mg/kg/day can be administered as partial doses multiple times in a day when it is determined that the entire daily dose does not need to be administered in one bolus or that it would be better to administer the daily dose in several increments.

One of ordinary skill in the art can determine the best route of administration of a pharmaceutical composition of the presently disclosed subject matter. For example, administration can be direct, enteral, or parenteral. Enteral includes, for example, oral and rectal administration. Parenteral includes, for example, intravenous administration. One of ordinary skill in the can determine the method and site of administration. For example, enteral, parental, direct, intravenous, or subcutaneous injection a composition comprising a protein agent (or biologically active fragments or homologs thereof), such as an antibody or cytokine, can be an effective treatment.

A compound or composition of the presently disclosed subject matter can be administered once or more than once. It can be administered once a day or at least twice a day. In some embodiments, a compound is administered every other day within a chosen term of treatment. In one embodiment, at least two compounds of the presently disclosed subject matter are used. One of ordinary skill in the art can determine how often to administer a compound of the presently disclosed subject matter, the duration of treatment, and the dosage to be used.

In some embodiments, the present disclosed subject matter provides compositions and methods involving the use of probiotics, prebiotics, or narrow spectrum antibiotics/anti-bacterial agents that are capable of restoring healthy mammalian bacterial gastrointestinal microbiota. The composition of the presently disclosed subject matter can further include growth factors, nutrient factors, pharmaceuticals, calcium-containing compounds, anti-inflammatory agents, antimicrobial agents, or any other substance capable of expediting or facilitating the treatment.

When two or more compounds are to be administered, they can be administered in the same pharmaceutical composition or in separate pharmaceutical compositions. When administered in separate pharmaceutical compositions, they can be administered simultaneously or one can be administered first. The amount of time between administration of the different compounds can vary and can be determined by one of ordinary skill in the art. For example, the two compounds could be administered up to 10 minutes apart, up to 30 minutes apart, up to 1 hour apart, etc. In some embodiments, one or more of the compounds can be administered more than once. In some embodiments, a compound is administered at least twice. In some embodiments, a compound is administered at least five times. In some embodiments, the method is useful for low dose treatment. In some embodiments, the method is useful for short-term treatment. In some embodiments, duration of treatment is from about 1-10, 1-9, 1-8, 1-7, 1-6, 1-5, 1-4, 1-3, or 1-2 days. In some embodiments, duration of treatment is from about 2-10, 2-9, 2-8, 2-7, 2-6, 2-5, 2-4, or 2-3 days. In some embodiments, duration of treatment is from about 3-10, 3-9, 3-8, 3-7, 3-6, 3-5, or 3-4 days. In some embodiments, duration of treatment is from about 4-10, 4-9, 4-8, 4-7, 4-6, or 4-5 days. In some embodiments, duration of treatment is from about 5-10, 5-9, 5-8, 5-7, or 5-6 days. In some embodiments, duration of treatment is from about 6-10, 6-9, 6-8, or 6-7 days. In some embodiments, duration of treatment is from about 7-10, 7-9, or 7-8 days. In some embodiments, duration of treatment is from about 8-10 or 8-9. In some embodiments, treatment is for about 1, 2, 3, 4, 5, 6, 7, 8, 9, or 10 days.

In some embodiments, a subject is treated daily during the treatment regimen when the duration is longer than one day. In some embodiments, the subject is treated every other day.

In some embodiments, the composition further comprises at least one additional therapeutic agent, e.g., an anesthetic, an analgesic, an antimicrobial, a steroid, a growth factor, a cytokine, an anti-inflammatory agent, or a combination thereof.

In some embodiments, the composition is for use in treating a subject who has a CDI. In some embodiments, the composition is for use in treating a subject with one or more increased risk factors for a CDI. In some embodiments, the composition is for use in treating or preventing recurring CDI.

II.B. Methods of Treatment and/or Prevention

In some embodiments, the presently disclosed subject matter provides methods for treating and/or preventing a C. difficile infection (CDI), and/or a symptom or pathology associated therewith, in a subject in need thereof. In some embodiments, the methods comprise, consist essentially of, or consist of administering to the subject a composition comprising, consisting essentially of, or consisting of at least one agent that inhibits an alpha 2 adrenergic receptor (ADRA2A) biological activity in the subject, wherein the administering is in an amount and via a route that treats and/or prevents the C. difficile infection and/or the symptom or pathology associated therewith in the subject. Any ADRA2A inhibitor can be used in the presently disclosed methods, including but not limited to those specifically disclosed herein. Exemplary such ADRA2A inhibitors include RX 821002, yohimbine, mianserin, mirtazapine, esmirtazapine, atipamezole, efaroxan, idazoxan, rauwolscine, spegatrine, dispegatrine, and phentolamine.

### II.C. Combination Treatments

In some embodiments, the presently disclosed subject matter provides methods for treatment and/or prevention that include use of active agents in addition to ADRA2A inhibitors. Such methods can include, for example, uses of an additional therapeutic agent that is selected from the group consisting of an anesthetic, an analgesic, an antimicrobial agent, a steroid, a growth factor, a cytokine, and an anti-inflammatory agent, or any combination thereof.

More particularly, PCT International Patent Application Publication No. WO 2018/022575 (incorporated herein by reference in its entirety) discloses that interleukin-33 (IL-33) and agonists/enhancers of IL-33 biological activities can be employed for treating and/or preventing CDI. Thus, in some embodiments the presently disclosed subject matter relates to combination therapies that employ one or more ADRA2A inhibitors in combination with one or more IL-33 agonists/enhancers. As previously described in PCT International Patent Application Publication No. WO 2016/057671 (incorporated herein by reference in its entirety), IL-25-induced eosinophilia is protective in CDI, and further that TLR2 antagonists would also be protective against C. difficile colitis (see Cowardin et al. (2016) The binary toxin

CDT enhances C. difficile virulence by suppressing protective colonic eosinophilia. Nature Microbiology 1(8):16108).

As disclosed in PCT International Patent Application Publication No. WO 2016/057671, IL-33 treatment protected from NAPI/027-associated mortality and weight-loss during C. difficile infection by skewing the immune response in the colon and by induction of regulatory T-cells. As such, IL-33 is a parallel pathway to IL-25 to induce protection from C. difficile and reduce mortality and severity of infection, as well as reduce signs associated with the infection. This pathway can be induced to treat and/or prevent C. difficile not only by stimulation of IL-33 (by microbiota transplants for example), or by small molecule agonists of the IL-33 signaling pathway, but also by TLR2 antagonists (since TLR2 activation potentiates the lethal effects of the C. difficile transferase (CDT) toxin of the epidemic strain of C. difficile). Therefore, the presently disclosed subject matter relates to use of ADRA2A inhibitors in addition to IL-33 agonists/enhancers to treat and/or prevent CDI, such as by stimulating IL-33 expression, levels, and activity as well as administering IL-33. While not wishing to be bound by any particular theory of operation, in some embodiments IL-33 treatment increases ST2⁺ regulatory T-cells during CDI.

It is also disclosed that IL-33 treatment acts as an anti-inflammatory agent. In some embodiments, it reduces detrimental colonic inflammation during CDI.

The presently disclosed subject matter further encompasses the use of microbiota transplants that stimulate IL-33 or molecules that activate the IL-33 pathway upstream or downstream of the IL-33 receptor.

Thus, in some embodiments the presently disclosed methods comprise, consist essentially of, or consist of administering to a subject in need thereof an effective amount of IL-33, or a biologically active fragment or homolog thereof. In some embodiments, the IL-33 protein comprises an amino acid sequence selected from any one of SEQ ID NOs: 16-27, and biologically active fragments and homologs thereof. The present invention further encompasses the use of active isoforms of IL-33. A summary of exemplary IL-33-related sequences that can be employed in the compositions and methods of the presently disclosed subject matter is present in PCT International Patent Application Publication No. WO 2016/057671.

The presently disclosed subject matter further provides for the use of IL-33 peptides with conservative amino acid substitutions that do not have a substantial effect on the activity described herein. For example, there can be up to 20 or up to about 10 conservative amino acid substitutions. In some embodiments, there are 1, 2, 3, 4, 5, 6, 7, 8, 9, or 10 substitutions.

Useful homologs of IL-33 and its active fragments can have varying degrees of homology provided that the homolog has similar activity to the polypeptide upon which it is based. In some embodiments, the homology can be at least about 75%, 80%, 85%, 90%, 95%, or at least 99% homology with the a reference IL-33 peptide or fragment.

Effective doses of IL-33 can vary depending on the age, sex, weight, and health of the subject and a dosage regimen or strategy can be developed by one of ordinary skill in the art. In one embodiment, an effective dose of IL-33 or a biologically active fragment or homolog thereof ranges from about 0.1 g/kg body weight to about 1,000 g/kg body weight. In some embodiments, the dosage is from about 1.0 g/kg body weight to about 500 g/kg body weight. In some embodiments, the dosage is from about 5.0 μg/kg body weight to about 200 μg/kg body weight. In a further aspect, the dosage is from about 10 μg/kg body weight to about 100 μg/kg body weight. In some embodiments, the dosage is selected from the group consisting of 1.0, 2.0, 3.0, 4.0, 5.0, 6.0, 7.0, 8.0, 9.0,10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 350, 375, 400, 425, 450, 475, and 500 μg/kg of body weight.

Clinical doses of proteins or biologically active fragments or homologs thereof are disclosed herein and are also known in the art. Doses can vary depending on the age, sex, weight, body surface area (BSA), and health of the subject, as well as the specific sign(s) of the infection being treated. Additionally, a higher dose may be used in some cases where an immediate effect is needed. Doses can be administered as a unit dose or the dose can be based on criteria such as those described above for body weight, etc. Doses can also be divided if administered more than once per day.

The number of doses of IL-33 to be administered can also vary depending on the age, sex, weight, body surface area (BSA), and health of the subject, as well as the specific sign(s) of the infection being treated. A pharmaceutical composition of the invention can be administered, for example, 1, 2, 3, 4, 5, 6, 7, 8, 9, or 10 times. In some embodiments, it can be administered up to 20 times. In some embodiments, it is administered once daily. In some embodiments it is administered more than once in a day. In some embodiments, it is administered once per week. In some embodiments, it is administered more than once per week. In some embodiments, it is administered once per month. In some embodiments, it is administered more than once per month.

The presently disclosed subject matter is useful for treating and/or preventing CDI and C. difficile colitis, including reducing mortality, preventing and/or inhibiting recurrent infection, inhibiting weight loss, inhibiting diarrhea, inhibiting colonic inflammation, and increasing colonic Foxp3+ ST2+ regulatory T cells during the infection. In some embodiments, the inflammation is neutrophil inflammation. In some embodiments, the inflammation is monocyte inflammation. In some embodiments, the inflammation is associated with both neutrophil and monocyte inflammation. In some embodiments, the compositions and methods of the presently disclosed subject matter are useful for replenishing IL-33 that has been depleted by antibiotic therapy.

In some embodiments, the invention encompasses the use of at least one additional therapeutic agent. Based on the teachings herein, one of ordinary skill in the art can determine which therapeutic agent(s) to administer, which can be, for example, an anesthetic, analgesic, antimicrobial, steroid, growth factor, cytokine, anti-inflammatory agent, drugs, probiotics, and pre-biotics. In some embodiments, the antimicrobial agent can be, for example, an antibacterial, antifungal, or antiviral agent. In some embodiments, an antibiotic useful for the present method includes, but is not limited to, vancomycin, fidaxomicin, metronidazole, nitazoxanide, and rifaximin.

The present invention further provides for combination therapies encompassing IL-33, IL-25, and regulation of TLRs.

It is disclosed herein that IL-33 comprises a parallel pathway to IL-25 for protection against C. difficile.

In some embodiments, the presently disclosed subject matter encompasses a combination therapy using IL-33 and an effective amount of an inhibitor of Toll-Like Receptor 2 (TLR2). In some embodiments, the inhibitor inhibits stimulation of TLR2 activity by C. difficile transferase toxin, thereby inhibiting the effects of C. difficile transferase toxin. In some embodiments, an inhibitor of the invention includes, but is not limited to, an antibody, antibody fragment, humanized antibody, monoclonal antibody, aptamer, phylomer, antisense oligonucleotide, nucleic acid, siRNA, protein, and a drug. In some embodiments, a monoclonal antibody of the invention is directed against TLR2. In some embodiments, a monoclonal antibody of the invention includes, but is not limited to, clone B4H2, clone C9A12, clone T2.5, clone mAb2616, clone TL2.1, and clone EPNCIR133.

In some embodiments, the compositions and methods of the invention are useful for protecting against CDI mediated virulence.

Similarly, PCT International Patent Application Publication No. 2021/097327 (incorporated herein by reference in its entirety) discloses that interleukin-13 (IL-13) and agonists/enhancers of IL-13 biological activities can be employed for treating and/or preventing CDI. As such, in some embodiments the presently disclosed subject matter provides methods for treating and/or preventing CDI, or a symptom or pathology associated therewith, in a subject in need thereof, the method comprising, consisting essentially of, or consisting of administering to the subject a composition that comprises a therapeutically effective amount of an ADRA2A inhibitor, optionally in combination with at least one IL-33 agonist/enhancer, and further comprising at least one agent that enhances a biological activity of interleukin-13 (IL-13) in the subject.

In some embodiments, the at least one agent that enhances a biological activity of IL-13 comprises an IL-13 peptide or a fragment or homolog thereof. In some embodiments, the IL-13 peptide is a peptide having an amino acid sequence that is at least 95% identical to any one of SEQ ID NOs: 9-11.

In some embodiments, the at least one agent that enhances an activity of IL-13 comprises an agent that blocks interleukin-13 receptor subunit alpha-2 (IL-13Ra2). In some embodiments, the agent that blocks IL-13Ra2 is an antibody that binds to IL-13Ra2, optionally wherein the antibody that binds to IL-13Ra2 is a monoclonal antibody.

In some embodiments, the administering results in a more rapid recovery from CDI as compared to the recovery expected in the absence of administering the composition to the subject. In some embodiments, the administering prevents a recurrence of CDI.

In some embodiments, the subject is a mammal. In some embodiments, the subject is a human. In some embodiments, the subject has a CDI. In some embodiments, the subject has one or more increased risk factors for a CDI, optionally wherein the one or more increased risk factors are selected from the group comprising hospitalization, antibiotic use, and increased age.

In some embodiments, administering the composition inhibits weight loss. In some embodiments, administering the composition inhibits diarrhea. In some embodiments, administering the composition inhibits colonic inflammation.

In some embodiments, the method further comprises administering at least one additional therapeutic agent to the subject, optionally wherein the composition comprises the at least one additional therapeutic agent. In some embodiments, said at least one additional therapeutic agent is selected from the group comprising an anesthetic, an analgesic, an antimicrobial agent, a steroid, a growth factor, a cytokine other than IL-13, and an anti-inflammatory agent.

In some embodiments, said at least one additional therapeutic agent comprises a cytokine other than IL-13, optionally an interleukin other than IL-13, further optionally wherein said at least one additional therapeutic agent comprises interleukin-4 (IL-4), interleukin-33 (IL-33), or interleukin-25 (IL-25). In some embodiments, said at least one additional therapeutic agent comprises an antimicrobial agent, optionally wherein said antimicrobial agent is selected from the group comprising an antibacterial agent, an antifungal agent, and an antiviral agent. In some embodiments, said antimicrobial agent comprises at least one antibiotic selected from the group comprising vancomycin, fidaxomicin, metronidazole, nitazoxanide, and rifaximin.

In some embodiments, the presently disclosed subject matter provides a composition for use in treating or preventing CDI, or a symptom or pathology associated therewith, wherein the composition comprises a therapeutically effective amount of at least one agent that enhances a biological activity of interleukin-13 (IL-13). In some embodiments, the agent that enhances a biological activity of IL-13 comprises an IL-13 peptide or a fragment or homolog thereof, optionally wherein the IL-13 peptide comprises an amino acid sequence that is at least 95% identical to any one of SEQ ID NOs: 9-11. In some embodiments, the agent that enhances a biological activity of IL-13 comprises an agent that blocks interleukin-13 receptor subunit alpha-2 (IL-13Ra2), optionally wherein said agent is an anti-IL-13Ra2 antibody. In some embodiments, the composition further comprises at least one additional therapeutic agent, optionally wherein said at least one additional therapeutic agent is selected from the group comprising an anesthetic, an analgesic, an antimicrobial agent, a steroid, a growth factor, a cytokine other than IL-13, and an anti-inflammatory agent.

In some embodiments, the presently disclosed subject matter provides a method of treating or preventing CDI, or a symptom or pathology associated therewith, in a subject in need thereof, the method comprising administering to the subject a composition that comprises a therapeutically effective amount of an interleukin-4 (IL-4) peptide, or a fragment or a homolog thereof, optionally wherein the IL-4 peptide comprises an amino acid sequence that is at least 95% identical to any one of SEQ ID NOs: 12-15. In some embodiments, the administering results in a more rapid recovery from a C. difficile infection as compared to the recovery expected in the absence of administering the composition to the subject. In some embodiments, the administering prevents a recurrence of a C. difficile infection.

In some embodiments, administering the composition inhibits weight loss. In some embodiments, administering the composition inhibits diarrhea. In some embodiments, administering the composition inhibits colonic inflammation.

In some embodiments, the method further comprises administering at least one additional therapeutic agent to the subject, optionally wherein the composition comprises the at least one additional therapeutic agent. In some embodiments, said at least one additional therapeutic agent is selected from the group comprising an anesthetic, an analgesic, an antimicrobial agent, a steroid, a growth factor, a cytokine other than IL-4, and an anti-inflammatory agent. In some embodiments, said at least one additional therapeutic agent comprises a cytokine other than IL-4, optionally an interleukin other than IL-4, further optionally wherein said at least one additional therapeutic agent comprises interleukin-13 (IL-13), interleukin-33 (IL-33), or interleukin-25 (IL-25). In some embodiments, said at least one additional therapeutic agent comprises an antimicrobial agent, optionally wherein said antimicrobial agent is selected from the group comprising an antibacterial agent, an antifungal agent, and an antiviral agent. In some embodiments, said antimicrobial agent comprises at least one antibiotic selected from the group comprising vancomycin, fidaxomicin, metronidazole, nitazoxanide, and rifaximin.

In some embodiments, the presently disclosed subject matter provides a composition for use in treating or preventing CDI, or a symptom or pathology associated therewith, wherein the composition comprises a therapeutically effective amount of an interleukin-4 (IL-4) peptide, or a fragment or a homolog thereof, optionally wherein the IL-4 peptide comprises an amino acid sequence that is at least 95% identical to any one of SEQ ID NOs: 12-15. In some embodiments, the composition further comprises at least one additional therapeutic agent, optionally wherein said at least one additional therapeutic agent is selected from the group comprising an anesthetic, an analgesic, an antimicrobial agent, a steroid, a growth factor, a cytokine other than IL-4, and an anti-inflammatory agent.

Summarily, the compositions and methods of the presently disclosed subject matter encompass, in some embodiments, combination treatments wherein at least one ADRA2A inhibitor is administered to a subject in need thereof in combination with one or more additional therapeutically active agents, wherein the one or more additional therapeutically active agents are selected from the group consisting of interleukin-33 (IL-33) and agonists/enhancers of IL-33 biological activities (including but not limited to an IL-33 polypeptide of any one of SEQ ID NOs: 16-27), IL-25 and/or biologically active fragments and derivatives thereof, regulators of Toll-Like Receptor (TLR) 2 gene products and other TLRs, enhancers/agonists of IL-13 biological activities, including but not limited to IL-13 peptides and/or fragments and/or homologs thereof, such as but not limited to peptides having an amino acid sequence that is at least 95% identical to any one of SEQ ID NOs: 9-11; agent that block interleukin-13 receptor subunit alpha-2 (IL-13Ra2) biological activities; IL-4 and/or biologically active fragments and derivatives thereof, including but not limited to IL-4 peptides that comprise, consist essentially of, or consist of an amino acid sequence that is at least 95% identical to any one of SEQ ID NOs: 12-15. It is noted that with respect to compositions and methods that employ more than one active agent (e.g., at least one ADRA2A inhibitor in combination with one or more interleukin-33 (IL-33) gene products and/or agonists/enhancers of IL-33 biological activities and/or one or more IL-25 gene products and/or biologically active fragments and derivatives thereof, and/or one or more regulators of Toll-Like Receptor (TLR) 2 gene products and/or other TLRs, and/or one or more enhancers/agonists of IL-13 biological activities, each individual active agent can be administered before, concurrently with, and/or after each other individual active agent, provided that at least one ADRA2A inhibitor is administered to the subject in need thereof at least once.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Materials and Methods for the EXAMPLES

Mice and Infections. All work with mice was conducted in compliance with ethical regulations for animal testing and research and all procedures approved by the Institute Animal Care and Use Committee (IACUC) of the University of Virginia (Charlottesville, Virginia, United States of America). Experiments were conducted with male, 8-12 week old C57BI/6 mice obtained from The Jackson Laboratory (Bar Harbor, Maine, United States of America). Mice were infected with the following protocol. An antibiotic cocktail was given in drinking water consisting of 45 mg/L Vancomycin (Mylan Pharmaceuticals Inc., Morgantown, West Virginia, United States of America), 35 mg/L Colistin (Sigma-Aldrich Corporation, St. Louis, Missouri, United States of America), 35 mg/L Gentamicin (Sigma-Aldrich), and 215 mg/L Metronidazole (Hospira, Inc., Lake Forest, Illinois, United States of America) on days 6, 5, and 4 prior to infection. Three days prior to infection (i.e., day −3), mice were switched back to untreated drinking water through the end of the experimental period. On the day prior to infection (day −1), mice were intraperitoneally injected with Clindamycin (cleocin phosphate) at 0.016 mg/g. Mice were then orally gavaged with 100-1000 CFU/mL of C. difficile R20291 (ribotype 027) spores or 10,000 C. difficile VPI spores in order to create comparable disease severity. For R20291 infections, data presented were representative of two experiments of 20 mice per group, whereas for VPI infections, data were representative of one experiment of 10 mice per group. After infection mice were monitored twice daily for signs of clinical disease. Mice were evaluated with respect to weight loss, coat condition, activity level, diarrhea, posture, and eye condition for a cumulative clinical score between 1 and 20. Weight loss and activity levels were scored between 0 and 4 with four being greater than or equal to 25% loss in weight. Coat condition, diarrhea, posture, and eye condition were scored between 0 and 3. Diarrhea scores were 1 for soft or yellow stool, 2 for wet tail, and 3 for liquid or no stool. Mice were euthanized if severe illness developed based on a clinical score≥14.

Pharmacological Treatments. All solutions were made freshly on their day of administration. 6-OHDA solutions were prepared by dissolving 6-OHDA hydrochloride (Sigma-Aldrich Catalog No. H4381) in PBS with 0.2% ascorbic acid (w/v) to prevent oxidation. For 6-OHDA treatment, mice were intraperitoneally injected on days −8,-7, and-6 prior to infection at 80 mg/kg for each mouse. In experiments with desipramine and 6-OHDA, desipramine hydrochloride (Sigma-Aldrich) was dissolved in PBS and administered intraperitoneally (i.p.) at 10 mg/kg 30 minutes prior to administration of 6-OHDA. For tests of blocking norepinephrine synthesis, nepicastat hydrochloride (Catalog No. HY-13289A; MedChemExpress, Monmouth Junction, New Jersey, United States of America) was crushed by mortar and pestle in ORA-PLUS® methylcellulose-based suspension. Mice were orally gavaged at with the nepicastat suspension on days −1, 0, and 1 post-infection. For testing the role of the alpha 2 adrenergic receptor, RX 821002 hydrochloride (Sigma-Aldrich) was dissolved in PBS and administered intraperitoneally at 10 mg/kg on day 0 and day 1 post-infection.

### Example 1

### Expression of ADRA2A is Downregulated in Severe CDI

Transcriptome microarray analysis was performed as described in Frisbee et al. (2019) IL-33 drives group 2 innate lymphoid cell-mediated protection during Clostridium difficile infection. Nature Communications 10: Article number 2712. Briefly, mice were infected with R20291 (CDT+) or R20291_cdtb (CDT−) and whole-cecal tissue transcriptomic analysis was performed on day 3 post infection. R20291 and R20291_Cdtb RNA samples were processed by the Affymetrix GENECHIP® WT PLUS Reagent Kit and hybridized to the Affymetrix Mouse Gene 2.0 ST GENECHIPR. The Affymetrix Mouse Transcriptome.CEL files were analyzed by the Bioinformatics Core at the University of Virginia. All preprocessing and analysis was done using R. Expression intensities were summarized, normalized, and transformed using Robust Multiarray Average algorithm (see Irizarry et al. (2003) Summaries of Affymetrix GeneChip probe level data. Nucleic Acids Research 31(4):e15). Probesets not mapping to an Entrez gene were excluded. For examining differential gene expression, a linear model was fit with empirical-Bayes moderated standard errors using the limma package in R.

FIG. 1 is a graph showing the results of the transcriptome microarray analysis. As shown in FIG. 1, expression of alpha 2 adrenergic receptors (ADRA2A) was downregulated in severe CDI.

### Example 2

### A Semi-permanent Method for Ablating Peripheral Catecholaminergic Neurons (6-OHDA)

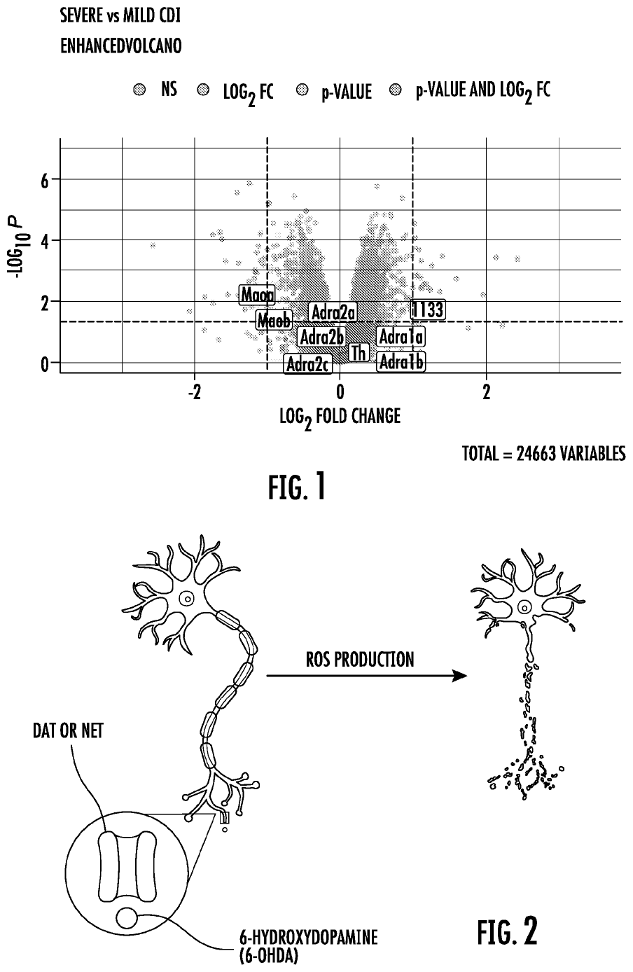

6-hydroxydopamine (6-OHDA) enters dopaminergic and noradrenergic neurons via dopamine transporters (DAT) and norepinephrine transporters (NET), respectively. 6-OHDA subsequently kills both types of neurons by ROS production and degeneration. FIG. 2 provides an exemplary schematic image of a semi-permanent method for ablating peripheral catecholaminergic neurons with 6-OHDA.

### Example 3

### A Mouse CDI Model

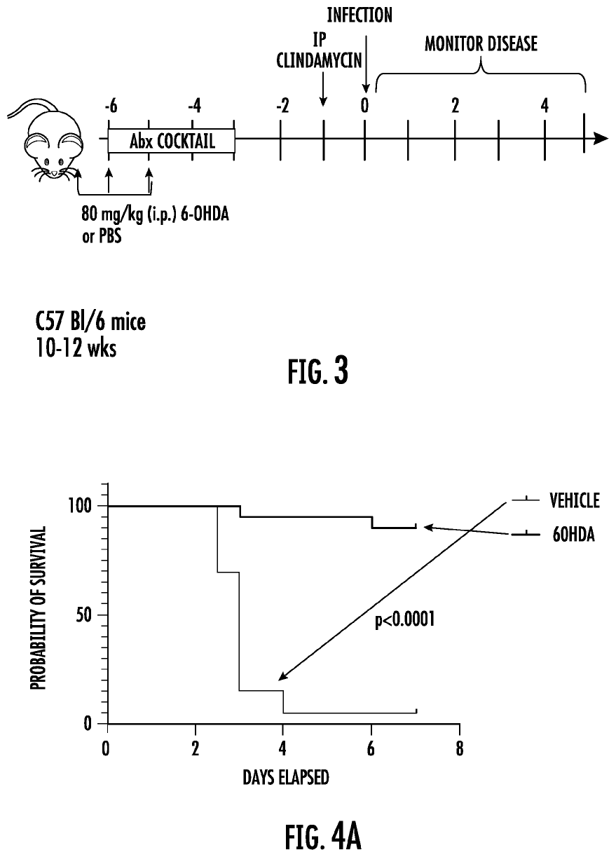

A mouse model of CDI was generated by administering 80 mg/kg of 6-OHDA intraperitoneally (i.p.) to 10-12 week old C57BI/6 mice at 7, 6, and 4 days prior to infection with C. difficile. At 6, 5, 4, and 3 days prior to infection, an antibiotic cocktail (Abx cocktail) was also administered to the mice, and at 1 day prior to infection, clindamycin was administered i.p. At day 0, the mice were infected with C. difficile, and disease was monitored over the next 5 days. The basic timeline is depicted in FIG. 3.

### Example 4

Sympathetic Nerve Ablation Protects Mice from CDI

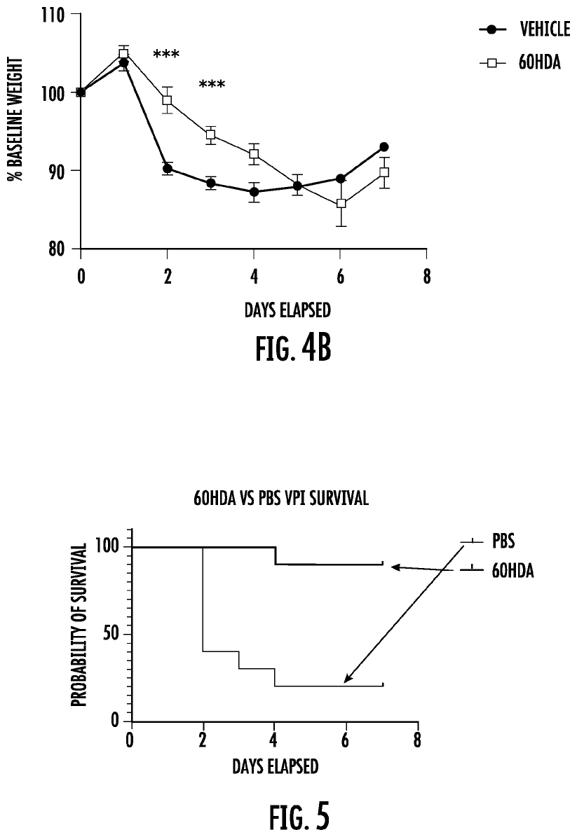

Using the model described in EXAMPLE 3 and FIG. 3, survival of mice treated with 6-OHDA was observed over 7 days. The results are shown in FIGS. 4A and 4B. As shown in FIG. 4A, 90% of mice treated with 6-OHDA (6OHDA) were still alive at day 7 post-infection, while only about 5% of mice that were treated with the vehicle (PBS) were alive past day 4 post-infection. This result was statistically significant (p<0.0001).

Weight loss was also tested in mice, and the results are shown in FIG. 4B. As shown therein, weight loss after sympathetic nerve ablation by 6OHDA (squares) was significantly (p<0.001) reduced at days 2 and 3 post-infection as compared to a vehicle control (circles).

FIG. 5 is a graph of probability of survival after sympathetic nerve ablation by 6OHDA (thicker black line) in the mouse model as compared to a PBS control (thinner black line). The data demonstrate that protection is likely not due to specific protection against binary toxin.

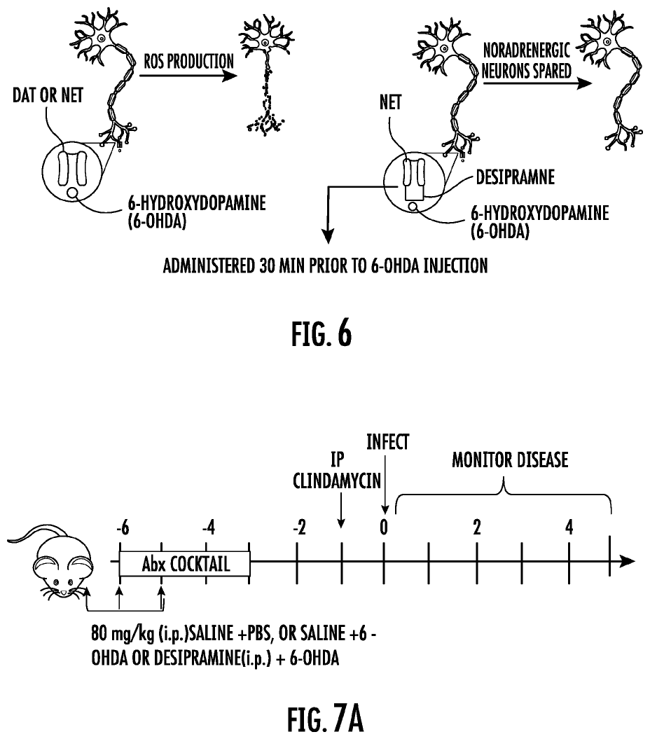

Blockade of NETs with NET-blocker desipramine precludes entry of 6-OHDA into noradrenergic neurons, sparing them from destruction. Dopaminergic neurons are still destroyed by 6-OHDA in the presence of desipramine, allowing for specific killing of these neurons while maintaining functional noradrenergic neurons. FIG. 6 is an exemplary schematic of desipramine sparing noradrenergic neurons from 6-OHDA mediated ablation.

### Example 5

### Noradrenergic Neurons Mediate CDI Pathogenesis

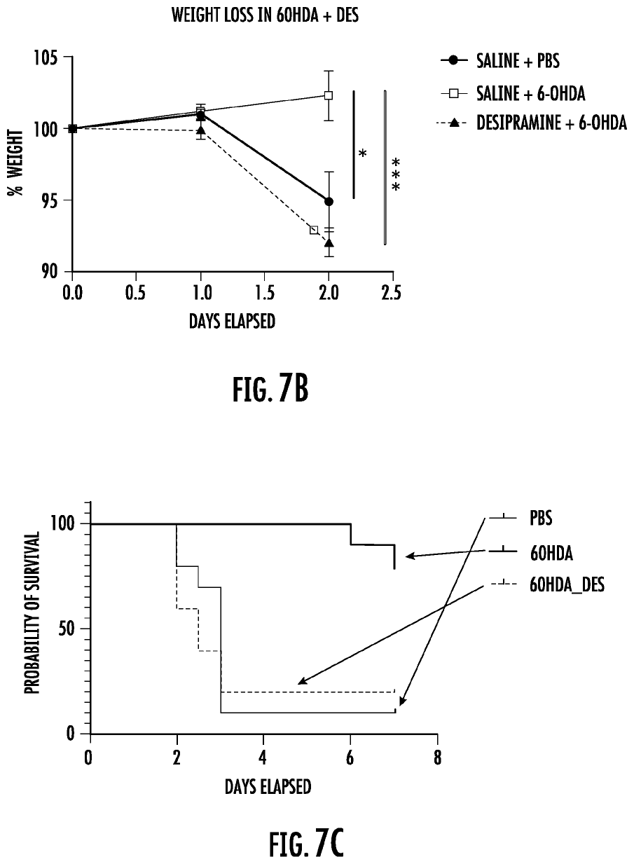

Whether noradrenergic neurons were involved in CDI pathogenesis was tested using the basic model depicted in FIG. 3. Mice were injected as shown therein but at 7, 6, and 4 days prior to infection with C. difficile, mice were administered saline plus PBS, saline plus 80 mg/kg 6-OHDA, or desipramine plus 6-OHDA. The timeline is shown in FIG. 7A.

From 0-2 days pose infection, weight loss was tested in each group. As shown in FIG. 7B, statistically significant weight losses were observed at day 2 post-infection in both the saline plus PBS group (p<0.05 vs. the saline plus 6-OHDA group) and the desipramine plus 6-OHDA group (p<0.001 vs. the desipramine plus 6-OHDA group).

Survival was also observed at days 0-7 post-infection, and as shown in FIG. 7C, survival of the desipramine plus 6-OHDA group largely reflected that of the PBS group in that greater than 80% of the mice in the group were dead by day 3 post-infection. However, through day 5, 100% of the 6-OHDA group were still alive, and at day 7 80% of the 6-OHDA group were still alive. This represented a statistically significant difference (p=0.001).

### Example 6

### 6-OHDA Mice are Characterized by Immune System Hypo-Responsiveness

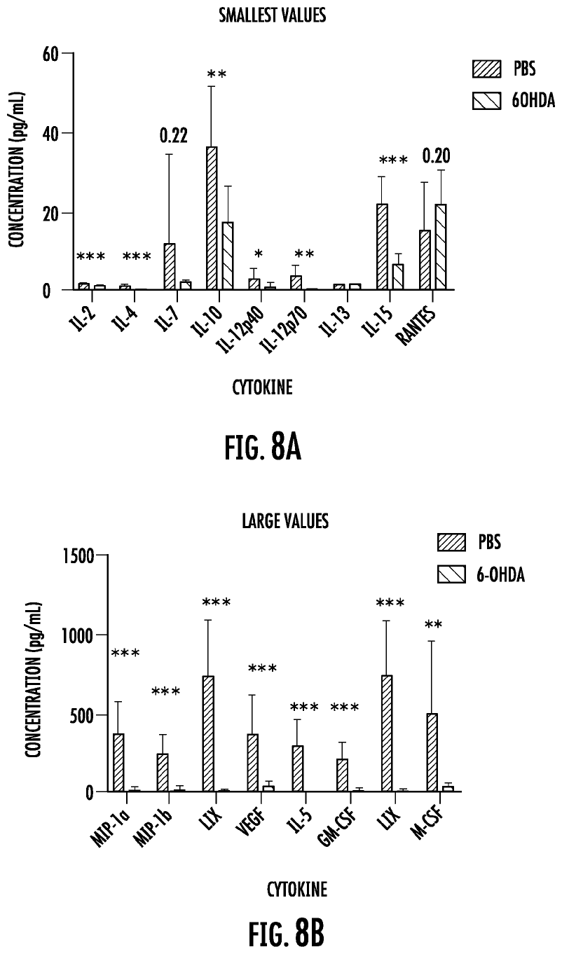

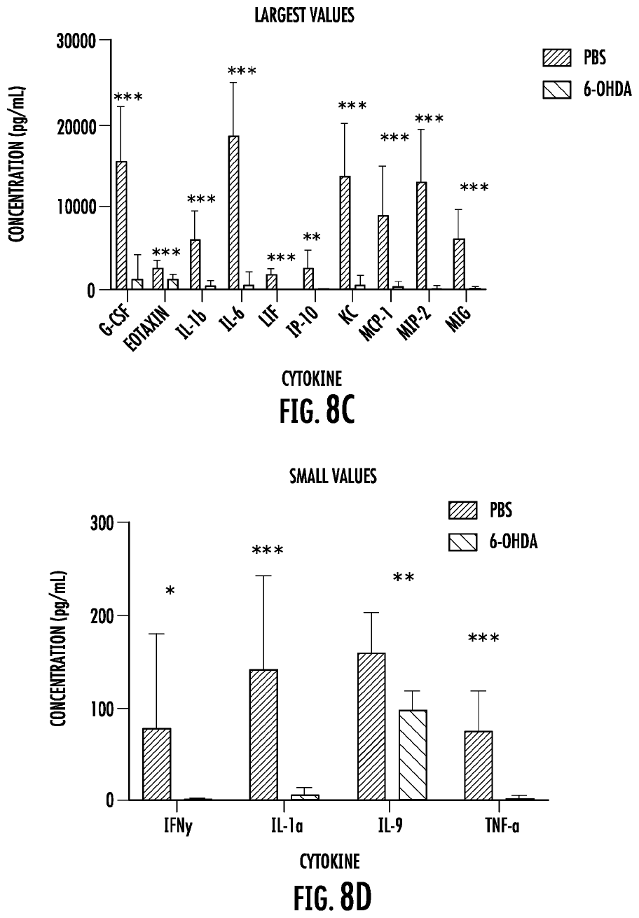

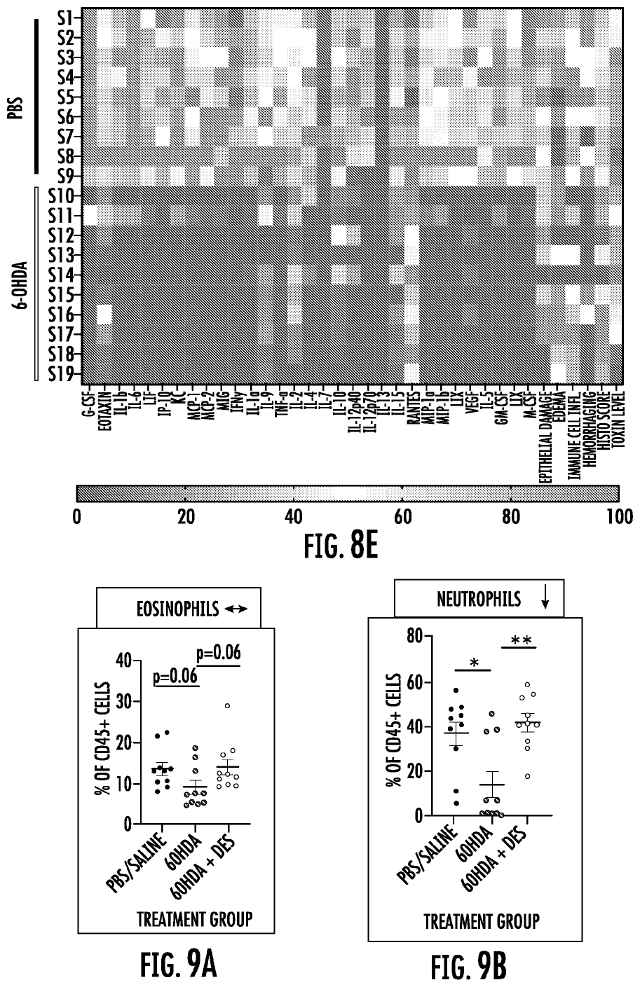

The levels of numerous cytokines were tested in PBS-treated mice and mice administered 6-OHDA. The results are shown in FIGS. 8A-8D.

Cytokines that were statistically significantly reduced in mice treated with 6-OHDA as compared with negative control mice treated with PBS included IL-2, IL-4, IL-10, IL-12 (p40 and p70 forms), IL-15 (FIG. 8A); MIP-1a, MIP-1b, LIX, VEGF, IL-5, GM-CSF, M-CSF (FIG. 8B); G-CSF, Eotaxin, IL-1b, IL-6, LIF, IP-10, KC, MCP-1, MIP2, MIG (FIG. 8C); IFNγ, IL-1a, IL-9, and TNFa (FIG. 8D).

Gene expression levels of various cytokines were also tested, and the results are shown in FIG. 8E, which shows a heat map of expression levels of cytokines in 6-OHDA-treated mice as compared to PBS-treated (i.e., negative control) mice.

### Example 7

### Noradrenergic Neurons are Required for CDI-induced Recruited Immunity

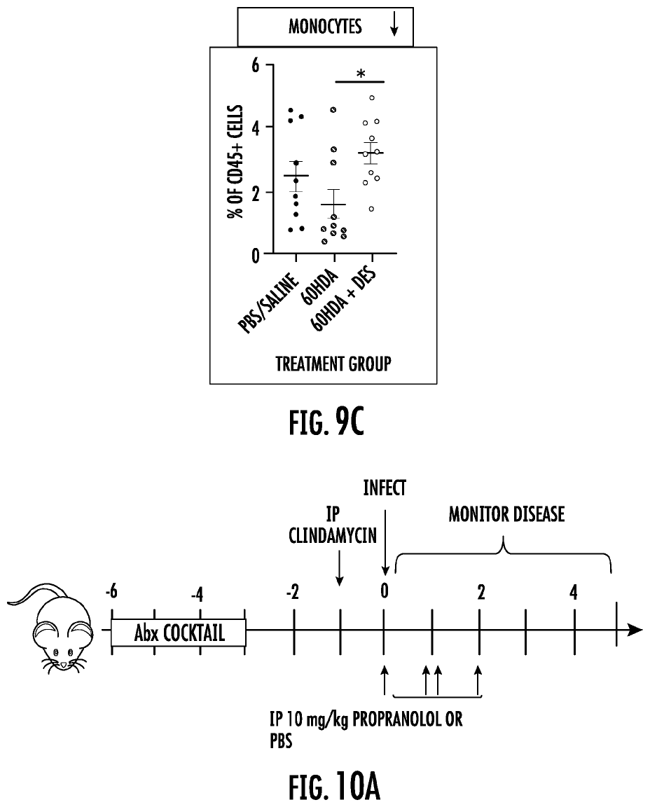

Whether noradrenergic neurons were required for CDI-induced recruited immunity was also tested, and the results are shown in FIGS. 9A-9C.

FIG. 9A is a plot showing eosinophil levels in negative control mice (PBS/Saline) vs. 6-OHDA-treated mice (6OHDA) vs. mice treated with 6OHDA+Desipramine (6OHDA+Des). The results showed that eosinophils were protective in CDI, although the decrease in eosinophils induced by 6-OHDA treated did not reach statistical significance.

FIG. 9B is a plot showing neutrophil levels in negative control mice (PBS/Saline) vs. 6-OHDA-treated mice (6OHDA) vs. mice treated with 6OHDA+Desipramine (6OHDA+Des). Neutrophil levels were significantly reduced by 6-OHDA treatment (p<0.05 vs. PBS/saline control), and treatment with desipramine negated this reduction (p<0.01 as compared to 6-OHDA alone). These results indicated that mice were able to survive CDI without a large entry of neutrophils, which is surprising given that neutrophils have been shown to be strongly implicated in protection against CDI.

FIG. 9C is a plot showing monocyte levels in negative control mice (PBS/Saline) vs. 6-OHDA-treated mice (6OHDA) vs. mice treated with 6OHDA+Desipramine (6OHDA+Des). Monocyte levels were not significantly reduced by 6-OHDA treatment (p<0.05 vs. PBS/saline control), but treatment with desipramine resulted in a statistically significant elevation of monocyte levels (p<0.05 as compared to 6-OHDA alone). These results indicated that monocytes were protective in CDI.

### Example 8

### The Beta 1 and 2 Blocker Propranolol Exacerbates CDI Disease Progression

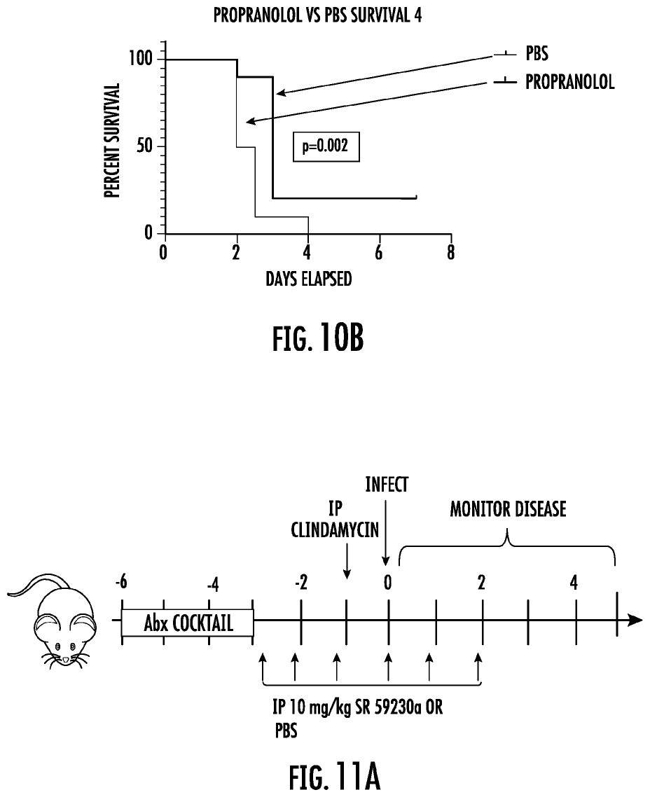

To test whether beta adrenergic neurons contributed to CDI disease progression, the beta 1 and 2 blocker propranolol was administered to mice as shown in the timeline depicted in FIG. 10A. The basic model is the same as for the previously described experiments, except that rather than treat with 6-OHDA, 10 mg/ml propranolol was administered to mice at days 0, 1 (two administrations), and 2 post-infection.

Percent survival was observed, and the results are shown in FIG. 10B. By day 4 post-infection, all propranolol-treated mice had died, whereas negative control (PBS) mice showed a slower mortality from day 2 on. In fact, about 25% of control mice survived past day 3 to at least day 7, and this difference was statistically significant (p=0.002).

### Example 9

### Beta 3 Blockade Exacerbates CDI Disease Progression

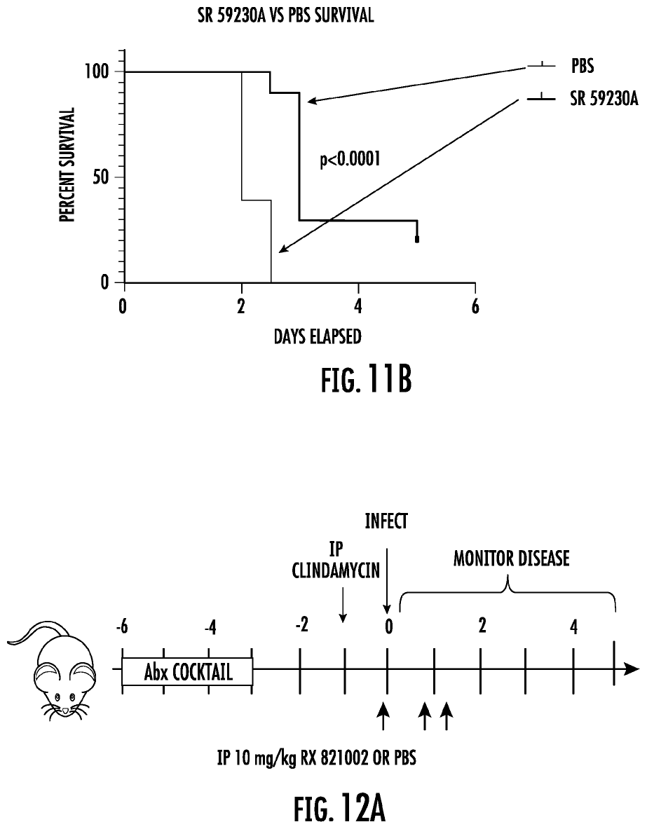

To test whether beta 3 adrenergic neurons contributed to CDI disease progression, the beta 3 blocker SR 59230a was administered to mice as shown in the timeline depicted in FIG. 11A. The survival curves are shown in FIG. 11B. Prior to day 3 post-infection, 100% of SR 59230A-treated mice had died, whereas at a similar time at which the last SR 59230A-treated mice had died, 90% of negative control mice were still alive. On days 3-5, a fraction of the negative control mice were also still alive. Thus, there was a statistically significant difference in percent survival between the two groups (p<0.0001).

### Example 10

### ADRA2A Inhibition Ameliorates CDI-Induced Mortality

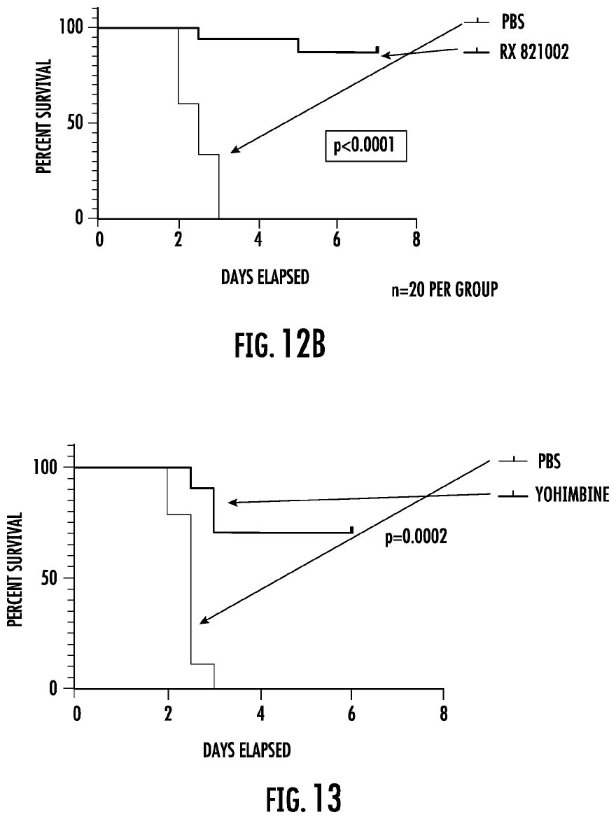

To further test whether ADRA2A inhibition affected CDI-induced mortality, the ADRA2A inhibitor 2-(3-methoxy-2H-1,4-benzodioxin-3-yl)-4,5-dihydro-1H-imidazole (RX 821002) was employed. The timeline for treatment was similar to that shown in FIG. 3 except that rather than treatment with 6-OHDA, mice were administered 10 mg·kg RX 821002 once immediately post-infection and twice on day 1 post-infection (see FIG. 12A). The survival curve is shown in FIG. 12B. As shown therein, there was a statistically significant difference in percent survival between the two groups (p<0.0001).

A similar experiment was performed using the Adra2A inhibitor yohimbine (methyl(1S,15R,18S,19R,20S)-18-hydroxy-1,3,11,12,14,15,16,17,18,19,20,21-dodecahydroyohimban-19-carboxylate). A survival curve for yohimbine-treated mice and negative control (PBS-treated) mice is shown in FIG. 13. Yohimbine provided a statistically significant amelioration of CDI induced mortality (p=0.0002).

### Example 11

### Colonization Does Not Result in a Difference in CDI Burden

C. difficile counts were quantified from cecal contents at 18 hours post-infection. Cecal contents were resuspended by mass in anaerobic PBS. Cecal contents were diluted, plated on BHI agar supplemented with 1% sodium taurocholate, 1 mg/mL D-cycloserine, and 0.032 mg/mL cefoxitin (Sigma-Aldrich), and anaerobically incubated at 37° C. overnight. Colony counts were made manually.

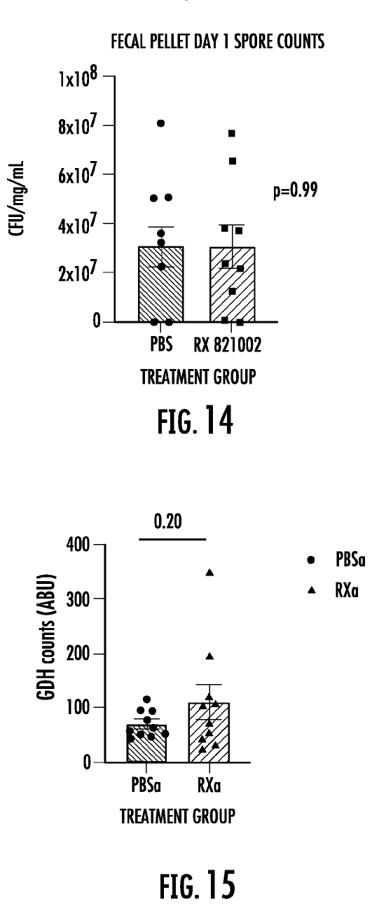

The results are presented in FIG. 14. As shown in FIG. 14, the negative control treatment group (PBS) and the RX 821002 treatment group (RX 821002) showed no difference in colony-forming units (CFU)/mg/ml (p=0.99).

### Example 12

### ADRA2A Inhibition Has No Effect on CDI Colonization

CDI colonization levels were also determined in PBS-treated mice (negative control) and mice treated with RX 821002. Briefly, C. difficile colonization in cecal contents of mice sacrificed 2 days post-infection was determined by a GDH ELISA from TECHLAB, Inc. (Blacksburg, Virginia, United States of America; Catalog No. TL5025). Prior to the ELISA, cecal contents were resuspended in PBS by mass. The GDH ELISA was performed according to the manufacturer's instructions.

The results are presented in FIG. 15. As shown therein, RX 821002 (RXa) treatment had no effect on CDI colonization as compared to a negative control (PBSa).

### Example 13

### ADRA2A Inhibition Reduces Cecal Toxin A/B

ADRA2A inhibition with RX 821002 was also tested for any affects on toxin A/B levels in mice. Briefly, C. difficile colonization in cecal contents of mice sacrificed 2 days post-infection was determined by a Toxin A/B ELISA from TECHLAB, Inc. (Catalog No. TL5025). Prior to the ELISA, cecal contents were resuspended in PBS by mass. The Toxin A/B ELISA was performed according to the manufacturer's instructions. Mice were treated with RX 821002 and toxin A/B levels in CDI cecal content were determined.

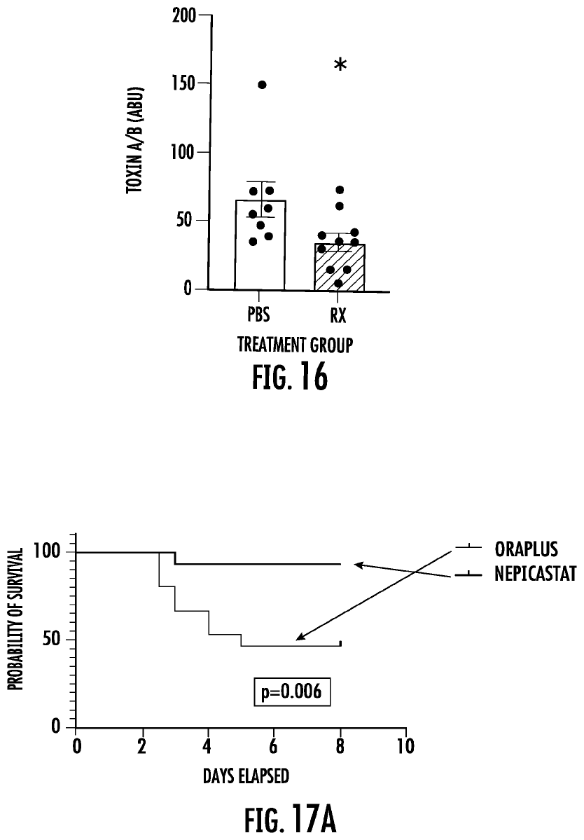

The results are shown in FIG. 16. As shown therein, RX 821002 (RX) treatment reduced toxin A/B levels in CDI cecal content.

### Example 14

### Norepinephrine Synthesis Blockade Ameliorates CDI-Induced Mortality

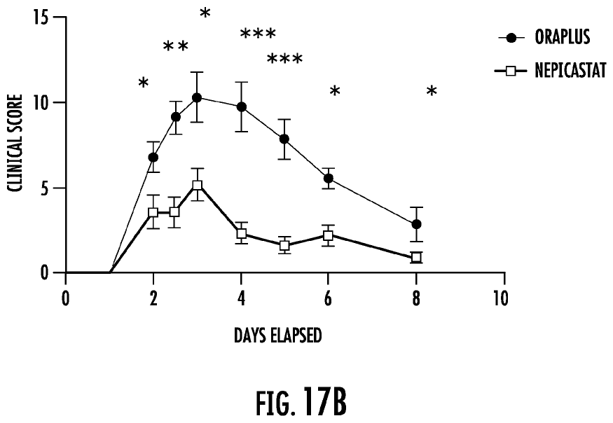

To determine if blocking norepinephrine synthesis influenced CDI, mice were administered nepicastat hydrochloride (Catalog No. HY-13289A; MedChemExpress, Monmouth Junction, New Jersey, United States of America) in ORA-PLUS® methylcellulose-based suspension on days −1, 0, and 1 post-infection. Survival and clinical scores (described herein above) were determined, and the results are presented in FIGS. 17A and 17B, respectively.

As shown in FIG. 17A, administration of nepicastat hydrochloride protected against CDI-induced mortality (p=0.006). Nepicastat-treated mice also had significantly better clinical scores at days 2-8 (0.001<p<0.05).

### Discussion of the EXAMPLES

To test the role of the sympathetic nervous system in C. difficile infection (CDI) disease progression, a mouse model of infection was employed. Prior to infection, mice are administered 6-hydroxydopamine (6-OHDA) to peripherally destroy sympathetic termini. 6-OHDA, administered on days 8, 7, and 6 prior to infection appreciably reduced the levels of norepinephrine in cecal tissue at 2 days post infection (see FIG. 3). Pretreatment with 6-OHDA strikingly ameliorated the incidence of mortality and, transiently, weight loss at the peak of infection (i.e., days 2-4). These data suggested that sympathetic nervous system activity was essential for the disease phenotype in CDI.

As 6-OHDA affects both dopaminergic and noradrenergic neurons, it is possible that both or a single neuronal subtype mediates disease. Desipramine, an inhibitor of the norepinephrine transporters (NETs), was used to isolate the role of noradrenergic neurons in driving disease as it spares noradrenergic but not dopaminergic neurons from destruction by 6-OHDA. Administration of desipramine 30 minutes prior to 6-OHDA prevented the protective effect of 6-OHDA on the incidence of mortality (see FIG. 6).

The sympathetic nervous system signals through five major adrenergic receptor subtypes: alpha 1, alpha 2, beta 1, beta 2, and beta 3. Administration of propranolol, an antagonist of beta 1 and 2 adrenergic receptors, or SR 59230A, an antagonist of beta 3 adrenergic receptors, increased the incidence of mortality in the presently disclosed CDI mouse model. Prazosin, an alpha 1 adrenergic antagonist, had little to no effect on the rate of mortality. Conversely, RX 821002, an alpha 2 adrenergic antagonist, drastically reduced the incidence of morality in the mouse model, phenocopying the effects of 6-OHDA. These data suggested that the sympathetic nervous system drove CDI disease via the alpha 2 adrenergic receptor.

To test the possibility that the sympathetic nervous system signals through the alpha 2 adrenergic receptor via norepinephrine, norepinephrine synthesis was inhibited in the CDI mouse model. Nepicastat, an inhibitor of dopamine beta hydroxylase (DBH), halts the conversion of dopamine to norepinephrine. As in the case with 6-OHDA and RX821002 treatment, the administration of nepicastat greatly reduced the incidence of mortality in the mouse model. This finding further supported the idea that the sympathetic nervous system drove disease via a norepinephrine-alpha 2 adrenergic receptor axis.

The sympathetic nervous system is closely apposed to immune cells in the intestine. To determine whether the sympathetic nervous system was necessary for a mounting of the inflammatory response, the infiltrating immune cell response in 6-OHDA-treated mice was measured by flow cytometry. Infiltrating neutrophils, and monocytes and eosinophils to a lesser extent, were less representative of the colonic immune cells (see FIG. 9). Likewise, lower amounts of cytokines (both inflammatory and anti-inflammatory) were measured in the cecal tissue of 6-OHDA-treated mice (see FIG. 8). These data suggested that the intestine was spared of inflammation in the absence of sympathetic neurons in spite of CDI infection.

It will be understood that various details of the presently disclosed subject matter may be changed without departing from the scope of the presently disclosed subject matter. Furthermore, the foregoing description is for the purpose of illustration only, and not for the purpose of limitation.

1. A method for treating and/or preventing a Clostridioides difficile (C. difficile) infection (CDI), and/or a symptom or pathology associated therewith, in a subject in need thereof, the method comprising, consisting essentially of, or consisting of administering to the subject a composition comprising, consisting essentially of, or consisting of at least one agent that inhibits an alpha 2 adrenergic receptor (ADRA2A) biological activity in the subject, wherein the administering is in an amount and via a route that treats and/or prevents the C. difficile infection and/or the symptom or pathology associated therewith in the subject.

2. The method of claim 1, wherein the at least one agent is selected from the group consisting of RX 821002, yohimbine, mianserin, mirtazapine, esmirtazapine, atipamezole, efaroxan, idazoxan, rauwolscine, spegatrine, dispegatrine, and phentolamine.

3. The method of claim 2, wherein the at least one agent is selected from the group consisting of RX 821002 and yohimbine.

4. The method of claim 1, further comprising administering to the subject at least one additional anti-C. difficile agent.

5. The method of claim 4, wherein the at least one additional anti-C. difficile agent is selected from the group consisting of an enhancer of an IL-13 biological activity, optionally wherein the enhancer of the IL-13 biological activity comprises, consists essentially of, or consists of an IL-13 peptide or a fragment or homolog thereof; an interleukin-13 receptor subunit alpha-2 (IL-13Ra2) inhibitor; an enhancer of an Interleukin-33 (IL-33) biological activity, optionally an IL-33 polypeptide or a biologically active fragment or homolog thereof; and combinations thereof.

6. The method of claim 5, wherein the IL-13Ra2 inhibitor is an antibody that binds to IL-13Ra2, optionally wherein the antibody that binds to IL-13Ra2 is a monoclonal antibody.

7. The method of claim 1, wherein the subject is a mammal, optionally a human.

8. The method of claim 7, wherein the human has one or more increased risk factors for CDI, optionally wherein the one or more increased risk factors for CDI are selected from the group consisting of hospitalization, antibiotic use, and increased age.

9. The method of claim 1, further comprising administering to the subject at least one additional therapeutic agent, optionally wherein the at least one additional therapeutic agent is selected from the group consisting of an anesthetic, an analgesic, an antimicrobial agent, a steroid, a growth factor, a cytokine other than IL-13, and an anti-inflammatory agent.

10. The method of claim 9, wherein said at least one additional therapeutic agent comprises a cytokine other than IL-13, optionally an interleukin other than IL-13, further optionally wherein said at least one additional therapeutic agent comprises interleukin-4 (IL-4), interleukin-33 (IL-33), or interleukin-25 (IL-25), or any combination thereof.

11. The method of claim 9, wherein said at least one additional therapeutic agent comprises an antimicrobial agent, optionally wherein said antimicrobial agent is selected from the group consisting of an antibacterial agent, an antifungal agent, and an antiviral agent.

12. The method of claim 11, wherein said antimicrobial agent comprises at least one antibiotic selected from the group consisting of vancomycin, fidaxomicin, metronidazole, nitazoxanide, and rifaximin.

13. The method of claim 4, wherein the at least one agent that inhibits an ADRA2A biological activity in the subject, the optional at least one additional anti-C. difficile agent, or both are administered at least twice to the subject.

14. The method of claim 1, wherein the symptom or pathology associated with the CDI is selected from the group consisting of weight loss, diarrhea, and colonic inflammation, optionally wherein the colonic inflammation is selected from the group consisting of neutrophil inflammation, monocyte inflammation, and eosinophile inflammation.

15. A composition for use in treating and/or preventing a Clostridioides difficile (C. difficile) infection and/or a symptom and/or pathology associated therewith, the composition comprising, consisting essentially of, or consisting of a therapeutically effective amount of at least one agent that inhibits an alpha 2 adrenergic receptor (ADRA2A) biological activity in the subject.

16. The composition for use of claim 15, wherein the at least one agent that inhibits the ADRA2A biological activity is selected from the group consisting of RX 821002, yohimbine, mianserin, mirtazapine, esmirtazapine, atipamezole, efaroxan, idazoxan, rauwolscine, spegatrine, dispegatrine, and phentolamine, optionally wherein at least one agent that inhibits the ADRA2A biological activity is selected from the group consisting of RX 821002 and yohimbine.

17. The composition for use of claim 15, wherein the composition further comprises a therapeutically effective amount of at least one agent that comprises an enhancer of an IL-13 biological activity, optionally wherein the enhancer of the IL-13 biological activity comprises, consists essentially of, or consists of an IL-13 peptide or a fragment or homolog thereof;
an interleukin-13 receptor subunit alpha-2 (IL-13Ra2) inhibitor; an enhancer of an Interleukin-33 (IL-33) biological activity, optionally an IL-33 polypeptide or a biologically active fragment or homolog thereof; and combinations thereof. In some embodiments, the IL-13Ra2 inhibitor is an antibody that binds to IL-13Ra2, optionally wherein the antibody that binds to IL-13Ra2 is a monoclonal antibody.

18. The composition for use of claim 17, wherein the enhancer of the IL-13 biological activity comprises, consists essentially of, or consists of an IL-13 peptide or a fragment or homolog thereof and/or the enhancer of the Interleukin-33 (IL-33) biological activity comprises, consists essentially of, or consists of an IL-33 polypeptide or a biologically active fragment or homolog thereof.

19. The method of claim 1, wherein the method or composition for use reduces mortality, prevents or inhibits recurrent infection, reduces weight loss, reduces diarrhea, and/or reduces colonic inflammation, optionally wherein the colonic inflammation is selected from the group consisting of neutrophil inflammation, monocyte inflammation, and eosinophil inflammation, when administered to a subject in need thereof as compared to what would have occurred had the subject not been administered the at least one agent that inhibits the ADRA2A biological activity or the composition.


SEQUENCE LISTING (27 sequences)

SEQ ID NO: 1 - DNA, 3879 bp, Homo sapiens
  source 1..3879: mol_type = other DNA
gagcagcagcagctccagctcggtgcagaagcccagcagccggcgtgccgccgcccggccactccagcgccttcttccccgccttgcgctcctgccccaactcgcgctgtcgtcggaccccggcccatccagcagcgctcggcgcccaccaggcggacgcccaggagaacccctgcctccgtcgcggctcctggagagctgatcgttcacctgccccggcccgcctgaggacgggggtgccttcatgcggcccccacactcctcaccccgccgccgccgccgtcccggagctccgcacagtgtgccccagccccagcagggcgcacaactttggaagtctcgcggcgctccgagaggcggcagagtccgcgccccagccccgggccgggccgggccagaaccgcagcgtctgggggaagccagagagtcggtaatcgcttcggggatgtaaggcgacagacataggacccccgagctcgcatcagcacccttcggctgcctcccggggtgggggcgggccccgcacacggtaagacctcttgctttcgctcaggctcaagattcaagatacagatattgatatgtatatatatatttaatttcctgtcatccttccaagttatcaggccaccgatgatttttgttctcccttcttgaagaataaatctctctttacccatcggctctccctactctctcccgccgcttagaaataaaacttggctgtattaggagctcggagcaagaaggcgcccaccgagagcgtctgaagcgcgagccaggcgcagttcgcgggacccgggccatgggccgctagcggtcctccagttcgggcccggcctccctgcggccccctccctatgtgagccgcagccaggcgagcggggcgccggaggaagaggaggacccacgggcgccgggccggaaggcagctggcagcaggcccaggccagcgggcgcccgcgttcatgttccgccaggagcagccgttggccgagggcagctttgcgcccatgggctccctgcagccggacgcgggcaacgcgagctggaacgggaccgaggcgccggggggcggcgcccgggccaccccttactccctgcaggtgacgctgacgctggtgtgcctggccggcctgctcatgctgctcaccgtgttcggcaacgtgctcgtcatcatcgccgtgttcacgagccgcgcgctcaaggcgccccaaaacctcttcctggtgtctctggcctcggccgacatcctggtggccacgctcgtcatccctttctcgctggccaacgaggtcatgggctactggtacttcggcaaggcttggtgcgagatctacctggcgctcgacgtgctcttctgcacgtcgtccatcgtgcacctgtgcgccatcagcctggaccgctactggtccatcacacaggccatcgagtacaacctgaagcgcacgccgcgccgcatcaaggccatcatcatcaccgtgtgggtcatctcggccgtcatctccttcccgccgctcatctccatcgagaagaagggcggcggcggcggcccgcagccggccgagccgcgctgcgagatcaacgaccagaagtggtacgtcatctcgtcgtgcatcggctccttcttcgctccctgcctcatcatgatcctggtctacgtgcgcatctaccagatcgccaagcgtcgcacccgcgtgccacccagccgccggggtccggacgccgtcgccgcgccgccggggggcaccgagcgcaggcccaacggtctgggccccgagcgcagcgcgggcccggggggcgcagaggccgaaccgctgcccacccagctcaacggcgcccctggcgagcccgcgccggccgggccgcgcgacaccgacgcgctggacctggaggagagctcgtcttccgaccacgccgagcggcctccagggccccgcagacccgagcgcggtccccggggcaaaggcaaggcccgagcgagccaggtgaagccgggcgacagcctgccgcggcgcgggccgggggcgacggggatcgggacgccggctgcagggccgggggaggagcgcgtcggggctgccaaggcgtcgcgctggcgcgggcggcagaaccgcgagaagcgcttcacgttcgtgctggccgtggtcatcggagtgttcgtggtgtgctggttccccttcttcttcacctacacgctcacggccgtcgggtgctccgtgccacgcacgctcttcaaattcttcttctggttcggctactgcaacagctcgttgaacccggtcatctacaccatcttcaaccacgatttccgccgcgccttcaagaagatcctctgtcggggggacaggaagcggatcgtgtgaggtttccgctggcgcccgcgtagactcacgctgactgcaggcagcggggggcatcgaggggtgcttagccccagggcactcagaaacccgggcgctgcctgctctgcgtttcctcgtctggggtggctctgcagcctcctgcgggcgggcgtctgctgctcctacaagggaagcttcttgctgccaggcccacacatccccagttgttggtttggccactcttgacctggagccatcttcctagtgggccacccctaatcactattgcttcctaaaggtattttcaccctcttcgcctggtacagccctcacagctcttcagagcaagcactggactacaagggcatggctcacaaaaggttaatggatgggggttacctagccctggctaattccccttccattcccaactctctctctctttttaaagaaaaatgctaagggcagccctgcctgccctccccatcccccgctgtaaatatacactatttttgatagcacacatggggcccccatatctcttggccttggttttgatgttgaaatcctggccttgggagagatgccttccaggcagacacagctgtctggttcaggccaagcccctttgcaatgcaagccctttctggtgttatgaagtccctctatgtcgtcgttttcaccagcaactggtgactgtcccttcgacacggacctgctttgagatttcctgacagggaaaagatttctgtccatttttttcctgtgcctaacagcataattgccttttcctatgtaaatattatgatggtggatcaagacataagtaaatgagcctttctgcctcacatcagccctgtgtataaagccattattctctgatgcactgtttgccccagtaactcactttaaaacctctctttccagtgttccctctctccctccagggccactgcttgaagaagaatatgtatgtttctatcttgtatgtctgtgtgcccctcctgccccgaaagtgctgactatggggaaatcttttagctgctgtttttagactccaaggagtggaaattatgtggaagaagcaaacctgatacaatttgcccaaggtaaacagtttgaaaagacaaatgggcctgccaaactgtacagtttcttccccaagagctgttaggtatcaaaatgttgtcctttcccccctccgtgcttttctggttgagatcatgtcattgatgaactgccaaagtcaggggaggagggcagagactttgtgtttacatctgcatttctacatgttttagacagagacaatttaaggcctgcactcttatttcactaaagaaaaactaatgtcagcacatgttgctaatgacagtggatttttttttaaataaaaaagtttacagatcaaatgtgaaataaatatgaatggagtggtcctcttgtctgttatctgagttttcaaaagctttaagactctgggaacatctgattttatggattttttaaaaataaaaaatgtacattataaaaa

SEQ ID NO: 2 - protein, 465 aa, Homo sapiens
MFRQEQPLAEGSFAPMGSLQPDAGNASWNGTEAPGGGARATPYSLQVTLTLVCLAGLLMLLTVFGNVLVIIAVFTSRALKAPQNLFLVSLASADILVATLVIPFSLANEVMGYWYFGKAWCEIYLALDVLFCTSSIVHLCAISLDRYWSITQAIEYNLKRTPRRIKAIIITVWVISAVISFPPLISIEKKGGGGGPQPAEPRCEINDQKWYVISSCIGSFFAPCLIMILVYVRIYQIAKRRTRVPPSRRGPDAVAAPPGGTERRPNGLGPERSAGPGGAEAEPLPTQLNGAPGEPAPAGPRDTDALDLEESSSSDHAERPPGPRRPERGPRGKGKARASQVKPGDSLPRRGPGATGIGTPAAGPGEERVGAAKASRWRGRQNREKRFTFVLAVVIGVFVVCWFPFFFTYTLTAVGCSVPRTLFKFFFWFGYCNSSLNPVIYTIFNHDFRRAFKKILCRGDRKRIV

SEQ ID NO: 3 - DNA, 3724 bp, Mus musculus
  source 1..3724: mol_type = other DNA
agcagcctcggctcccgcttccgtgcagacgcccagcagccggcgcacaccgcccagccacccagcgcctccagctccaagtcctgcggtcctgcgaaaactcacgcggccctccgacccccgccgggtctgtggcgctcagcgcccacccagatcgcggctagcagccacccttggctccgccgcggctcctggagagccggtcgctcacctagcttggccctgaggaccgaggtggtggtggtgggggtgcggcttcatgcggcccccacacccttcaccccaccgccgccgcccccgagctctgtgtacagagtcccagccccagcagagcgcacaactttggaagtcccgtggggctcctagaggaggcagagtccgcgccttcgccggaggtccggcccagccctcgccaccccgcctggagggtagcgacggagttggaaatctcttcgagggtataagaagacagacctaggactccgagcccacaccaacacccctccgctgcctcttggggtaagacttcatgctttcgctcagcttaagaatcaaggtatagatgactagctaaatgcatttgtataaatttctgacatctttccaaatcatcaggccaccgataatttcttttctcttttcttgaagaataaaatctctaaccaccagctggccctactcgatcctgccgcttagaaataaaacttggctgtgtttgaggagctgggagagagaaagcacccacccggagcgtcggaaggacgaaccggtgccagttcgcgggctccagtccatggaccactagcggtccccagcttgggcccgagctccctgcggccctcttccttatgtgaggcgcggcgggacgaaccccgcaccggaggaagaggaggacccacggacgcggggccggaaggcagctggcagcaggcccacgagaaccagcacccgcgttcatgttccgccaggagcagccgctggccgagggcagctttgcgcccatgggctcactgcagccggatgccggcaacagcagctggaacgggaccgaagcgcccggaggcggcacccgagccaccccttactccctgcaggtgacactgacgctggtttgcctggctggcctgctcatgctgttcacagtatttggcaacgtgctggttattatcgcggtgttcaccagtcgcgcgctcaaagctccccaaaacctcttcctggtgtccctggcctcagcggacatcctggtggccacgctggtcattcccttttctttggccaacgaggttatgggttactggtactttggtaaggtgtggtgtgagatctatttggctctcgacgtgctcttttgcacgtcgtccatagtgcacctgtgcgccatcagccttgaccgctactggtccatcacgcaggccatcgagtacaacctgaagcgcacgccgcgtcgcatcaaggccatcattgtcaccgtgtgggtcatctcggctgtcatctccttcccgccactcatctccatagagaagaagggcgctggcggcgggcagcagccggccgagccaagctgcaagatcaacgaccagaagtggtatgtcatctcctcgtccatcggttccttcttcgcgccttgcctcatcatgatcctggtctacgtgcgtatttaccagatcgccaagcgtcgcacccgcgtgcctcccagccgccggggtccggacgcctgttccgcgccgccggggggcgccgatcgcaggcccaacgggctgggcccggagcgcggcgcgggtcccacgggcgctgaggcggagccgctgcccacccagcttaacggtgccccgggggagcccgcgcccgccgggccccgcgatggggatgcgctggacctagaggagagttcgtcgtccgagcacgccgagcggcccccggggccccgcagacccgaccgcggcccccgagccaagggcaagacccgggcgagtcaggtgaagccgggggacagtctgccgcggcgcgggcccggggccgcggggccgggggcttcggggtccgggcacggagaggagcgcggcgggggcgccaaagcgtcgcgctggcgcgggaggcaaaaccgggagaaacgcttcacgttcgtgctggcggtggtgatcggcgtgttcgtggtgtgttggtttccgttctttttcacctacacgctcatagcggtcggctgcccggtgcccagccagctcttcaacttcttcttctggttcggctactgcaacagctcgctgaaccctgttatctacaccatcttcaaccacgacttccgacgcgccttcaagaagatcctctgccgtggggacagaaaacgcatcgtgtgagcgcatgggccttgccctgcgtgcagacaatggtcagacgcaggccctgggcttcgagggggtgcgtctacgcatgcagcccctagcactctgaaaccccggacctacccagctgggagggagctccgtggcctttgagaccttttgttacaatgtgtaaagttctggctgagagggacacatgagcccttttgctggttgttgtggttgttcccagcccacagccagtttcctggtgggctacccctgatcagtatggtttcctgatgatattgtcatatccccaagtctgactctcccaagctcttcagagcaagcacagtactagagagggcatggctgccaaagggttaatgaaccaaggtttcctctagtcctggctaattatctcctccacacccccttccttaaataaataaataaagatcattcagggcggtcctgcctgccctcccttctccccactgtaaatatacattatttttgatagcatactttgggttcttcagtgtcggccttgatgcatattggaatcatggctgtggagatgccctccagtcagacagtgggctatctggtccagaccacgctcctttgcaatgcaagcccctttctagtgttaagttgctctcagtgcttttcagcagtaactggtgactgtcccctggacacggacctgctttgacatttcctgacagggaagggatttctgctcattttcccctgtgcctaactgcataattgccttttcctatgtaaatattacagcgatggaccaaggcagaaggaaatgaagcctctgccttgcatcagccccgtgtataaagccattatctgctgaggcaccgcttgccccagtaactcactttagaactgacttttcttccgttctcccctcctctggggcttgaagaagagcgtgtatgtttctatcttgtctgtgcgccgccccctcccccagaaagtgctgactaaggggaatcttttagctgctgtttttagacacccaggaggggaaattatgtggaagaaaccaacccgatacaatttgccaaggtaaacagtgtgaggagacaaatgggctgccacactgtatggagccagccccaagagctcttgggtatcaaagtgttacctccccttgctgtcctggctgagatcatgtgactactgaattgccaaagtcagggggtaggcggagacttcgtgtttacaacctggtttctacttattttaggcagactgtggaaggcctgtgatctgatttcacgagagaaaaactaaatgtcagcacttgttgctcgtgacggtggattttttttaaataaaagtttacagatcaaatgtgaaataaacatgaattaagtggg

SEQ ID NO: 4 - protein, 465 aa, Mus musculus [sequence omitted]

SEQ ID NO: 5 - DNA, 2814 bp, Rattus norvegicus
  source 1..2814: mol_type = other DNA
atgttccgccaggagcagccgctggccgagggcagctttgcgcccatgggctccctgcagccggatgccggcaatagcagctggaacggcaccgaggcgcccggaggcggcacccgggccaccccttactccctgcaggtgacgctgacgctggtgtgcctggctggcctgctcatgctgttcaccgtgtttggcaacgtgctggttattatcgcagtgttcaccagccgcgcgctcaaagcgccccagaacctcttcctggtgtctctggcctcagcggacatcctggtggccacgctggtcattcccttttctttggccaacgaggttatgggctactggtactttggtaaggtgtggtgcgagatctacttggccctcgacgtgctcttttgcacgtcgtccatagtgcacctgtgcgccatcagccttgaccgctactggtccatcacgcaggccatcgagtacaacctgaagcgcacgccgcggcgcatcaaggccatcattgtcactgtgtgggtcatctcggccgtcatctccttcccgccactcatctccatagagaagaagggcgctggcggcgggcagcagccggccgagccgagctgcaagattaacgaccagaagtggtatgtcatctcgtcgtccatcggctccttcttcgcgccttgcctcatcatgatcctggtctacgtgcgtatctaccagatcgccaagcgtcgcacccgcgtgccgcccagtcgccggggtccggacgcctgttccgcgccgccggggggcgccgatcgcaggcccaacgggctgggcccggagcgcggcgctggtaccgcgggcgcggaggccgagccgctgcccacccagcttaacggcgccccgggggagcccgcgcccacccggccgcgcgacggggatgcgctggacctagaggagagttcgtcgtccgagcacgccgagcggccccaggggcccggcaaacccgagcgcggtccccgggccaagggcaagaccaaggcgagccaggtgaaaccgggggacagtctgccgcggcgcgggcccggggctgcggggccgggggcttcggggtccgggcagggagaggagcgtgccgggggcgccaaagcgtcgcgctggcgcgggaggcagaaccgcgagaaacgcttcacgttcgtgctggcggtggtgatcggcgtgttcgtggtgtgttggttcccgttctttttcacctacacgctcatagcggtcggctgcccggtgccctaccagctcttcaacttcttcttctggttcggctactgcaacagctcgctgaaccctgttatctacaccattttcaaccacgacttccgccgcgccttcaagaagatcctctgccgtggggacagaaagcgcatcgtgtgagcgctcgggctgtgccctgcttacagaccctggtcagatgcaggccctgggcttcgagggggcgcgtctacgcatgcagcccctagcactctgaaaccctggaccttcctgctctgtgtaatacccagtttggaggggacttggtggcttttgagaccttttgttaccatgtgtaaagttctggctgagagggacatatgcgcctttttgctggttgttgtggttgttcccagcccacagccagtttcctggtgggctacccccgatcagtattgtttcctgacgatattgccatcttctccccgagtctgactctcccaagctcttcagagcaagcacagtactagagagggcatgactgccaaagggttaatgaaccaaggtttcctagccctggctaattgtcacttccctccttccccccctttaaaaatatcattcagggcagccctgcctgccctccgtaccctccactgtaaatatacattatttttgatagcacactttgtgttcctcagaattggccttgtcgcgatattggaatcccggctgtggggatgccctccagtcacagacagtgggctatctggtccagaccaacgtcctttgcaatgcaagtccctttctagtgttattacatcgtcgctctcagtacttttcagcagtaactggtaactgtcccctggacacggacctgctttgacatttcctgagagggaagggatttctgttcattttccccctgtgcctaacagcataattgccttttcctatgtaaatattacagcgatggaccaagacagaaggaaatgaaccttctgcctggccccagccctgtgtataaagccattattcgctgaggcaccacttgccccagtaactcactttagaactgacgctgcggttctcctctcctctggggccaaggcttgaagaagaacgggtgggtgtgcttgtttctgtcttgtgtgtgtgccgcccctcatccagaaagtgctgagtaaggggaatcttttagcagctgtttttagacacacaggaggggaaattatgtggaagaaacctacccgatacaatttgccaaggtaaacagtgtgaggagacaaacagtccgccacactgttcagcgccagccccaagagctcttgggtatcaaagtgttacctccccttgcttttctggctgagatcacgtcaccagtgaattgccaaagtcagggagtaggcggagaattggtgtttacaacctggtttctacttagtttaggcggactgtggaaggcctgcgatctgtttttcctgagagaaaaacctaaatgtcagcacttgttgctcatgatggtggatttttttttaaaataaaagtttacagatcaaatgtgaaataaacatgaattaagtgataaaaaaaaaaaaa

SEQ ID NO: 6 - protein, 465 aa, Rattus norvegicus
MFRQEQPLAEGSFAPMGSLQPDAGNSSWNGTEAPGGGTRATPYSLQVTLTLVCLAGLLMLFTVFGNVLVIIAVFTSRALKAPQNLFLVSLASADILVATLVIPFSLANEVMGYWYFGKVWCEIYLALDVLFCTSSIVHLCAISLDRYWSITQAIEYNLKRTPRRIKAIIVTVWVISAVISFPPLISIEKKGAGGGQQPAEPSCKINDQKWYVISSSIGSFFAPCLIMILVYVRIYQIAKRRTRVPPSRRGPDACSAPPGGADRRPNGLGPERGAGTAGAEAEPLPTQLNGAPGEPAPTYRRDGDALDLEESSSSEHAERPQGPGKPERGPRAKGKTKASQVKPGDSLPRRGPGAAGPGASGSGQGEERAGGAKASRWRGRQNREKRFTFVLAVVIGVFVVCWFPFFFTYTLIAVGCPVPYQLFNFFFWFGYCNSSLNPVIYTIFNHDFRRAFKKILCRGDRKRIV

SEQ ID NO: 7 - DNA, 3872 bp, Pan troglodytes
  source 1..3872: mol_type = other DNA
cagctccagctcggtgcagaagcccagcagccgacgtgccgccgcccggccactccagcgccttcttccccgccttgcgctcctgccccaactcgcgctgtcgtcggaccccggcccatccagcagcgctcggcgcccaccaggcggacgcccaggagaacccctgcctccgtcgcggctcctggagagctgatcgttcacctgccccggcccgcctgaggacgggggtgccttcatgcggcccccacactcctcaccccgccgccgccgccgcccccgagctccgcacagtgcgccccagccccagcagggcgcacaactttggaagtctcgcggcgctccgagaggcggcagagtccgcgccccagccccgggccgggccgggccagaaccgcagcgtctgggggaagccagagagtcggtaatcgcttcggggatgtaaggcgacagacataggaccccgagctcgcatcagcacccctcggctgcctcccggggtgggggcgggccccgcacacggtaagacctcttgctttcgctcaggctcaagattcaagatatagatattgatatgtatatatatatttaatttcctgtcatccttccaagttatcaggccaccgatgatttttgttctcccttcttgaagaataaatctctctttacccatcggctctccctactctctcccgccgcttagaaataaaacttggctgtattaagcgctcggagcaagaaggcgcccaccgagagcgtctgaagcgcgagccaggcgcagttcgcgggacccgggccatgggccgctagcggtcctccagttcgggcccggcctccctgcggccccctccctatgtgagccgcagccaggcgagcggggcgccggaggaagaggaggacccacgggcgccgggccggaaggcagctggcagcaggcccaggccagcgggcgcccgcgttcatgttccgccaggagcagccgttggccgagggcagctttgcgcccatgggctccctgcagccggacgcgggcaacgcgagctggaacgggaccgaggcgccggggggcggcgcccgggccaccccttactccctgcaggtgacgctgacgctggtgtgcctggccggcctgctcatgctgctcaccgtgttcggcaacgtgctcgtcatcatcgccgtgttcacgagccgcgcgctcaaggcgccccaaaacctcttcctggtgtctctggcctcggccgacatcctggtggccacgctcgtcatccctttctcgctggccaacgaggtcatgggctactggtacttcggcaaggcgtggtgcgagatctacctggcgctcgacgtgctcttctgcacgtcgtccatcgtgcacttgtgcgccatcagcctggaccgctactggtccatcacacaggccatcgagtacaacctgaagcgcacgccgcgccgcatcaaggccatcatcatcaccgtgtgggtcatctcggccgtcatctccttcccgccgctcatctccatcgagaagaagggcggcggcggcggcccgcagccggccgagccgcgctgcgagatcaacgaccagaagtggtacgtcatctcgtcgtgcatcggctccttcttcgctccctgcctcatcatgatcctggtctacgtgcgcatctaccagatcgccaagcgtcgcacccgcgtgccacccagccgccggggtccggacgccgtcgccgcgccgccggggggcgccgagcgcaggcccaacggtctgggccccgagcgcagcgcgggcccggggggcgcagaggccgaaccgctgcccacccagctcaacggcgcccctggcgagcccgcgccggccgggccgcgcgacgccgacgcgctggacctggaggagagctcgtcttccgaccacgccgagcggcctccagggccccgcagacccaagcgcggtccccggggcaaaggcaaggcccgagcgagccaggtgaagccgggggacagcctgccgcggcgcgggccgggggcgacggggatcgggacgccggctgcagggccgggggaggagcgcgtcggggctgccaaggcgtcgcgctggcgcgggcggcagaaccgcgagaagcgcttcacgttcgtgctggccgtggtcatcggagtgttcgtggtgtgctggttccccttcttcttcacctacacgctcacggccgtcgggtgctccgtgccacgcacgctcttcaagttcttcttctggttcggctactgcaacagctcgctgaacccggtcatctacaccatcttcaaccacgatttccgccgcgccttcaagaagatcctctgtcggggggacaggaagcggatcgtgtgaggtttccgctggcgcccgcgtagactcacgctgactgcaggcagcggggggcatcgaggggtgctcagcctcagggcactcagaaacccgggcgctgcctgctctgcgtttcctcgtctggggtggctctgcagcctcctgcgggcgggcgtctgctgctcctacaagggaagcttcctgctgccaggcccacacatccccagttgttggtttggccactcttgacctggagccatcttcctagtgggccacccctaatcagtattgcttcctaaaggtattttcaccctcttcgcctggtacagccctcacagctcttcagagcaagcactggactacaagggcatggctcacaaaaggttaatggatgggggttacctagctctggctaattccccttccattcccaacgctctctctctctctttttaaagaaaaatgctaagggcagccctgcctgccctccccaccccccactgtaaatatacactatttttgatagcacacatggggcccccatatctcttggccttggttttgatgttgaaatcctggccttgggagagatgccttccaggcagacacagctgtctggttcaggccaagcccctttgcaatgcaagccctttctggtgttatgaagtccctctatgtcgtcgttttcaccagcaactggtgactgtcccttcgacatggacctgctttgagatttcctgacagggaaaagatttctgtccatttttttcctgtgcctaacagcataattgccttttcctatgtaaatattatgatggtggatcaagacataagtaaatgagcctttctgcctcacatcagccctgtgtataaagccattattctctgatgcactgttcgccccagtaactcactttaaaacctctctttccagtgttccctctctccctccagggccactgcttgaagaagaatatgtatgtttctatcttgtatgtctgtgtgcccctcctgccccgaaagtgctgactatggggaaatcttttagctgctgtttttagactccaaggagtggaaattatgtggaagaagcaaacctgatagaatttgcccaaggtaaacagtttgaaaagacagatgggcctgccaaactgtacagttccttccccaagagctgttaggtatcaaaatgttgtcctttcccccctccctgcttttctggttgagatcatgtcattgatgaactgccaaagtcaggggaggagggcagagaccttgtgtttacatctgcgtttctacatgttttagacagagacaatttaaggcctgcactcttatttcactaaagaaaaactaatgtcagcacatgttgctaatgacagtggattttttttaaataaaaaagtttacagatcaaatgtgaaataaatatgaatggagtggtcctcttgtctgttatctgagttttcaaaagctttaagactctgggaacatctgattttatggattttttaaaaataaaaaatgtaccttataaaaa

SEQ ID NO: 8 - protein, 465 aa, Pan troglodytes
MFRQEQPLAEGSFAPMGSLQPDAGNASWNGTEAPGGGARATPYSLQVTLTLVCLAGLLMLLTVFGNVLVIIAVFTSRALKAPQNLFLVSLASADILVATLVIPFSLANEVMGYWYFGKAWCEIYLALDVLFCTSSIVHLCAISLDRYWSITQAIEYNLKRTPRRIKAIIITVWVISAVISFPPLISIEKKGGGGGPQPAEPRCEINDQKWYVISSCIGSFFAPCLIMILVYVRIYQIAKRRTRVPPSRRGPDAVAAPPGGAERRPNGLGPERSAGPGGAEAEPLPTQLNGAPGEPAPAGPRDADALDLEESSSSDHAERPPGPRRPKRGPRGKGKARASQVKPGDSLPRRGPGATGIGTPAAGPGEERVGAAKASRWRGRQNREKRFTFVLAVVIGVFVVCWFPFFFTYTLTAVGCSVPRTLFKFFFWFGYCNSSLNPVIYTIFNHDFRRAFKKILCRGDRKRIV

SEQ ID NO: 9 - protein, 146 aa, Homo sapiens
MHPLLNPLLLALGLMALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 10 - protein, 81 aa, Homo sapiens
MVWSINLTAGMYCAALESLINVSGCSAIEKTQRMLSGFCPHKVSAGQFSSLHVRDTKIEVAQFVKDLLLHLKKLFREGQFN

SEQ ID NO: 11 - protein, 131 aa, Mus musculus
MALWVTAVLALACLGGLAAPGPVPRSVSLPLTLKELIEELSNITQDQTPLCNGSMVWSVDLAAGGFCVALDSLTNISNCNAIYRTQRILHGLCNRKAPTTVSSLPDTKIEVAHFITKLLSYTKQLFRHGPF

SEQ ID NO: 12 - protein, 153 aa, Homo sapiens [sequence omitted]

SEQ ID NO: 13 - protein, 137 aa, Homo sapiens
MGLTSQLLPPLFFLLACAGNFVHGHKCDITLQEIIKTLNSLTEQKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 14 - protein, 136 aa, Homo sapiens
MGLTSQLLPPLFFLLACAGNFVHGHKCDITLQEIIKTLNSLTEQKTLCTELTVTDIFAASKQHACLPTSRHTGTICRPPSARVPSTSTRLQSPERTQLRRKPSAGLRLCSGSSTATMRRTLAAWVRLHSSSTGTSS

SEQ ID NO: 15 - protein, 139 aa, Mus musculus
MGLNPQLVVILLFFLECTRSHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESSTSLKDFLESLKSIMQMDYS

SEQ ID NO: 16 - protein, 270 aa, Homo sapiens
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 17 - protein, 264 aa, Homo sapiens
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 18 - protein, 229 aa, Homo sapiens
MYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 19 - protein, 228 aa, Homo sapiens
MKPKMKYSTNKISTAKWKNTASKALCFKLGKSQQKAKEVCPMYFMKLRSGLMIKKEACYFRRETTKRPSLKTGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 20 - protein, 228 aa, Homo sapiens
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 21 - protein, 222 aa, Homo sapiens
MKPKMKYSTNKISTAKWKNTASKALCFKLGSRKHKRHLVLAACQQQSTVECFAFGISGVQKYTRALHDSSITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 22 - protein, 180 aa, Homo sapiens
MKPKMKYSTNKISTAKWKNTASKALCFKLGKYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 23 - protein, 160 aa, synthetic construct
MSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 24 - protein, 159 aa, synthetic construct
SITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 25 - protein, 144 aa, Homo sapiens
MKPKMKYSTNKISTAKWKNTASKALCFKLGNKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 26 - protein, 266 aa, Mus musculus
MRPRMKYSNSKISPAKFSSTAGEALVPPCKIRRSQQKTKEFCHVYCMRLRSGLTIRKETSYFRKEPTKRYSLKSGTKHEENFSAYPRDSRKRSLLGSIQAFAASVDTLSIQGTSLLTQSPASLSTYNDQSVSFVLENGCYVINVDDSGKDQEQDQVLLRYYESPCPASQSGDGVDGKKVMVNMSPIKDTDIWLHANDKDYSVELQRGDVSPPEQAFFVLHKKSSDFVSFECKNLPGTYIGVKDNQLALVEEKDESCNNIMFKLSKI

SEQ ID NO: 27 - protein, 158 aa, Mus musculus
SIQGTSLLTQSPASLSTYNDQSVSFVLENGCYVINVDDSGKDQEQDQVLLRYYESPCPASQSGDGVDGKKVMVNMSPIKDTDIWLHANDKDYSVELQRGDVSPPEQAFFVLHKKSSDFVSFECKNLPGTYIGVKDNQLALVEEKDESCNNIMFKLSKI